(19) United States
(12) Patent Application Publication
(10) Pub. No.: US 2026/0201335 A1
(43) Pub. Date: Jul. 16, 2026

(54) A Method to Generate Cardiac Pericytes from Human Induced Pluripotent Stem Cells

(71) Applicant: The Board of Trustees of the Leland Stanford Junior University, Stanford, CA (US)

(72) Inventors: Joseph C. Wu, Palo Alto, CA (US); Mengcheng Shen, Redwood City, CA (US)

(21) Appl. No.: 19/135,756

(22) PCT Filed: Dec. 5, 2023
(86) PCT No.: PCT/US2023/082544

§ 371 Date: Jun. 4, 2025

Related U.S. Application Data
(60) Provisional application No. 63/430,916, filed on Dec. 7, 2022.

(51) Int. Cl. C12N 5/077; G01N 33/50

(52) U.S. Cl. CPC C12N 5/0657; G01N 33/5044; C12N 2501/135; C12N 2501/15; C12N 2501/155; C12N 2501/415; C12N 2501/727; C12N 2503/02; C12N 2506/45

(57) ABSTRACT

Methods are provided for the step-wise generation of epicardial cells and cardiac pericyte cells. Also provided are methods for generating pure populations of such cell types and derivatives thereof. The instant disclosure also provides methods of screening for cellular responses of the generated cell types and derivatives thereof. Treatment methods making use of the generated cell types and derivatives thereof are also provided. The instant disclosure also provides systems, compositions, and kits for practicing the methods of the disclosure.

### CROSS REFERENCE TO OTHER APPLICATIONS

This application claims the benefit of U.S. Provisional Application No. 63/430,916, filed Dec. 7, 2023, the contents of which are hereby incorporated by reference in its entirety.

### STATEMENT REGARDING FEDERALLY SPONSORED RESEARCH OR DEVELOPMENT

This invention was made with government support under contract HL113006 awarded by the National Institutes of Health. The government has certain rights in the invention.

### BACKGROUND

Cardiac pericytes (CPs), a major mural cell type maintaining homeostasis, integrity, and perfusion of the coronary microvasculature, remain the most enigmatic and underappreciated cell population in the heart. Accumulating evidence suggests that CPs play a key role in cardiovascular complications such as coronary vasospasm, no-reflow post myocardial infarction, and cancer drug-induced cardiotoxicity. However, the lack of unequivocal cell markers and specific tools for characterization, lineage tracing, and conditional targeting of CPs has precluded a comprehensive understanding of their pathogenic role in coronary microvascular dysfunction.

### SUMMARY

Methods are provided for producing mesodermal cell types, including methods of differentiation to epicardial cells, and to cardiac pericytes. Also provided are methods of screening for cellular responses and treating a subject for a condition using the produced cell types, populations of cell types, and/or terminally differentiated cells and tissues. The instant disclosure also provides systems and kits for producing mesodermal cell types, including epicardial cells; and cardiac pericytes; and/or screening for cellular responses and/or treating subjects with such mesodermal cell types.

Aspects of the method relate to producing mesodermal cell types and populations of mesodermal cell types, including epicardial cells; and cardiac pericytes, from pluripotent progenitor cells. In some embodiments, pluripotent progenitor cells are contacted in culture with a step-wise series of induction media to differentiate the starting cell population through mesodermal intermediate cell types into epicardial cells, which can be efficiently differentiated into cardiac pericytes in medium comprising an effective dose of PDGF-BB. The described methods, and individual steps thereof, may be performed independently or in a combination with and of the other described methods, and individual steps thereof.

An advantage of the methods of the disclosure is the high efficiency of induction of the produced epicardial cells, where, for example, greater than 90%, greater than 95%, greater than 98%, up to 99% or more of the cells in the population are induced to a WT1+ cell. An advantage of the methods of the disclosure is the production of mature epicardial cells, which express one or more of ALDH1A2, UPK3B and ANXA8, and which may express all of the ALDH1A2, UPK3B and ANXA8.

The methods produce a substantially pure population of cardiac pericytes, for example, greater than 90%, greater than 95%, greater than 98%, up to 99% or more of the cells in the population. Pericytes may be characterized as CD45 CD56 CD31 CD146+CD34 cells. The cells can express one or more of PDGFRB, RGS5, CSPG4, ANPEP, MCAM, ACTA2.

Aspects of the disclosure relate to screening mesoderm progenitors and/or differentiated mesoderm cell types derived or produced according to the methods described herein for a cellular response. In certain aspects, a method of screening mesoderm progenitors and/or differentiated mesoderm cell types for a cellular response may include contacting a population of mesoderm progenitors and/or differentiated mesoderm cell types with a pharmacological agent and evaluating the population of cells for a cellular response induced by the pharmacological agent. In certain aspects, the screening may be in vitro screening and the contacting may be performed in vitro. In certain aspects, the screening may be in vivo screening and the contacting may be performed by administering the pharmacological agent to a host animal that contains the population of cells.

Aspects of the disclosure relate to screening an animal for a phenotype wherein the host animal has been administered a genetically modified population of mesoderm progenitors and/or differentiated mesoderm cell types derived or produced according to the methods described herein. In certain aspects, the genetically modified population of mesoderm progenitors and/or differentiated mesoderm cell types derived or produced according to the methods described herein may include a genetic modification in at least one genetic locus. In certain aspects, the genetically modified population of mesoderm progenitors and/or differentiated mesoderm cell types derived or produced according to the methods described herein may include a genetic modification in at least one genetic locus resulting in disruption or deletion of at least one gene. In certain aspects, the host animal may be evaluated or a detectable phenotype induced by the administered population of cells.

Aspects of the disclosure relate to methods of treating a subject for a condition through the administration of mesoderm progenitors and/or differentiated mesoderm cell types derived or produced according to the methods described herein. In certain aspects, the method of treating a subject for a condition through administration of cells derived according to the methods as described herein may further include co-administration with at least one pro-survival or pro-engraftment factor. In certain aspects, the cells administered to a subject may be genetically modified at least one genetic locus.

Aspects of the disclosure include kits for the production, derivation, purification, and use of mesoderm progenitors and/or differentiated mesoderm cell types that include one or more induction compositions and/or one or more specific binding agents and/or combinations thereof. In certain aspects, such kits may or may not include one or more cell types described herein.

Aspects of the disclosure include systems for the production, derivation, purification, and use of mesoderm progenitors and/or differentiated mesoderm cell types that include one or more components configured to administer one or more induction compositions and/or one or more specific inducing agents and/or one or more specific binding agents and/or combinations thereof. In certain aspects, such systems are configured to administer such compositions and/or agents at specific amounts or for specific periods of time according to the methods described herein.

### BRIEF DESCRIPTION OF THE DRAWINGS

The invention is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity. Included in the drawings are the following figures.

FIG. 1. Phenotypic characterization and functional assessment of stepwise differentiated iPSC-cardiac pericytes. A, A schematic showing stage-specific inhibition and activation of morphogens to generate pure epicardial cells (EPI) from human induced pluripotent stem cells (iPSCs). Representative bright-field images for each stage of cell differentiation are demonstrated. MPS, mid-primitive streak; LPM, lateral plate mesoderm; SM, splanchnic mesoderm; ST, septum transversum; PEO, pre-epicardial organ. B, Immunofluorescent images (ZO1 and WT1), flow cytometry (WT1) graphs (n=10 iPSC lines, 5M/5F), and quantitative data showing EPI induction efficiency by stepwise (i) and GiWiGi (ii) protocols. C, Quantitative reverse transcription PCR (RT-qPCR) results showing expression levels of canonical (WT1, TBX18, and TCF21) and mature (ALDH1A2, UPK3B, and ANXA8) markers of EPIs derived by both protocols. D, Single-cell ATAC sequencing (scATAC-seq) of EPIs generated by both GiWiGi (ii) and stepwise (iii) protocols are projected to that of human fetal heart cell clusters (i). Dotted frames indicate the EPI cluster in the human fetal heart SCATAC-seq UMAP. E, Immunoblots showing time-dependent changes in cardiac pericytes (CP) and smooth muscle cell (SMC) markers during differentiation. iPSC-SMCs were used as a control. F, Flow cytometry and quantitative data (n=5, 2M/3F) showing iPSC-CP yields with and without exogenous platelet-derived growth factor (PDGF)-BB. G, A heatmap showing transcriptomic similarities of pericyte markers between primary and iPSC-CPs by RT-qPCR. H, Bright field and immunofluorescent images of iPSC-derived SMCs, cardiac fibroblasts (CFs), and endothelial cells (ECs). SM-MHC, smooth muscle-myosin heavy chain; NG2, neural/glial antigen 2. I, Calcium imaging using Fura-2 AM to quantitatively compare carbachol-induced intracellular calcium increases in iPSC-SMCs and iPSC-CPs. J, Tubular networks formed by iPSC-CPs, iPSC-ECs, and in combination in vitro (n=2, 1M/1F). iPSC-CPs were fluorescently labeled with Calcein Red-Orange AM. K, Vasculatures formed by iPSC-ECs alone (1.2×10⁶ cells/Matrigel plug) or in combination with iPSC-CPs (1.0×10⁶ iPSC-ECs and 0.2×10⁶ iPSC-CPs/Matrigel plug) after being implanted in male immunodeficiency NOD SCID mice (n=3 per group) for 7 days. Antibodies targeting iPSC-ECs and iPSC-CPs are human-specific. L, Dose-response curves for (i) iPSC-CMs, iPSC-CPs, and iPSC-ECs (n=3, 1M/2F per group) and (ii) primary and iPSC-CPs (n=2, 1M/1F per group) after 72 hr of sunitinib (0 μM-100 μM) treatment using a PrestoBlue viability assay. M, Bright-filed images showing the cell morphology of primary and iPSC-CPs after 72 hr of vehicle, sunitinib (5 μM), and sunitinib (5 μM)/thalidomide (1 μM) treatment. N, BrdU incorporation assays showing suppressed iPSC-CP proliferation by sunitinib (n=5, 2M/3F per group) in a dose-dependent manner. O, A heatmap showing gene clustering patterns of iPSC-CPs treated with vehicle, sunitinib (5 μM), and sunitinib (5 μM)/thalidomide (1 μM) for 72 hr. Key changes of hallmarks in each cluster are highlighted. P, Gene ontology pathway analysis of iPSC-CPs treated with sunitinib versus other conditions. GraphPad Prism 9 was used for statistical analysis. All data are presented as mean±sem. Statistical significance was assessed using a non-parametric statistical procedure (Wilcoxon signed-rank test for 2 groups and Kruskal-Wallis test for >2 groups). *p<0.05; *p<0.01; *p<0.001; *p<0.0001. ns indicates not significant.

### DETAILED DESCRIPTION OF THE EMBODIMENTS

Before the present methods and compositions are described, it is to be understood that this invention is not limited to particular method or composition described, and as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supercedes any disclosure of an incorporated publication to the extent there is a contradiction.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

It must be noted that as used herein and in the appended claims, the singular forms “a”, “an”, and “the” include plural referents unless the context clearly dictates otherwise. Thus, for example, reference to “a cell” includes a plurality of such cells and reference to “the peptide” includes reference to one or more peptides and equivalents thereof, e.g. polypeptides, known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### Definitions

The terms “treatment”, “treating”, “treat” and the like are used herein to generally refer to obtaining a desired pharmacologic and/or physiologic effect. The effect can be prophylactic in terms of completely or partially preventing a disease or symptom(s) thereof and/or may be therapeutic in terms of a partial or complete stabilization or cure for a disease and/or adverse effect attributable to the disease. The term “treatment” encompasses any treatment of a disease in a mammal, particularly a human, and includes: (a) preventing the disease and/or symptom(s) from occurring in a subject who may be predisposed to the disease or symptom(s) but has not yet been diagnosed as having it; (b) inhibiting the disease and/or symptom(s), i.e., arresting development of a disease and/or the associated symptoms; or (c) relieving the disease and the associated symptom(s), i.e., causing regression of the disease and/or symptom(s).

The terms “recipient”, “individual”, “subject”, “host”, and “patient”, are used interchangeably herein and refer to any mammalian subject for whom diagnosis, treatment, or therapy is desired, particularly humans. “Mammal” for purposes of treatment refers to any animal classified as a mammal, including humans, domestic and farm animals, and zoo, sports, or pet animals, such as dogs, horses, cats, cows, sheep, goats, pigs, camels, etc. In some embodiments, the mammal is human.

The terms “pluripotent progenitor cells”, “pluripotent progenitors”, “pluripotent stem cells”, “multipotent progenitor cells” and the like, as used herein refer to cells that are capable of differentiating into two or more different cell types and proliferating. Non limiting examples of pluripotent precursor cells include but are not limited to embryonic stem cells, blastocyst derived stem cells, fetal stem cells, induced pluripotent stem cells, ectodermal derived stem cells, endodermal derived stem cells, mesodermal derived stem cells, neural crest cells, amniotic stem cells, cord blood stem cells, adult or somatic stem cells, neural stem cells, bone marrow stem cells, bone marrow stromal stem cells, hematopoietic stem cells, lymphoid progenitor cell, myeloid progenitor cell, mesenchymal stem cells, epithelial stem cells, adipose derived stem cells, skeletal muscle stem cells, muscle satellite cells, side population cells, intestinal stem cells, pancreatic stem cells, liver stem cells, hepatocyte stem cells, endothelial progenitor cells, hemangioblasts, gonadal stem cells, germline stem cells, and the like.

Of interest herein are pluripotent progenitors having the capacity to generate mesodermal cell types or derivatives thereof, particularly induced pluripotent stem cells (iPSC). Pluripotent progenitors not naturally having the capacity to generate mesodermal cell types or derivatives thereof may be dedifferentiated to a cell type having such capacity by methods well-known in the art, including, e.g., those described below for the production of induced pluripotent cells.

Aspects of the disclosure include methods for deriving mesodermal cell types from pluripotent progenitor cells. Pluripotent progenitors of the instant disclosure may be acquired from any convenient source, including but not limited to newly derived from a subject of interest or tissue specimen or other cellular sample, obtained from a public repository, obtained from a commercial vendor, and the like. In some instances, pluripotent cells of interest include human cells including but not limited to, e.g., human embryonic stem cells, human induced pluripotent stem cells, human fetal stem cells, and the like.

In some instances, pluripotent progenitor cells of the subject disclosure may be unmodified such that the cells have not been genetically or otherwise modified from their natural state prior to modification according to the methods described herein. In other instances, pluripotent progenitor cells of the subject disclosure may be unmodified such that the cells have been genetically or otherwise modified from their natural state prior to modification according to the methods described herein. Modification of pluripotent progenitors and derived mesodermal cell type is described in further detail elsewhere herein.

The term “mesodermal cell types” as used herein refers to cells of the entire mesodermal linage and thus encompasses cells of the developing embryo that are non-ectodermal, non-endodermal, and non-germline. Mesodermal cell types, as used herein may refer to mesodermal progenitors and/or differentiated mesodermal cell types.

The terms “mesodermal progenitors” and “mesoderm progenitors” are used interchangeably herein and generally refer to precursor and/or progenitor cells capable of giving rise to one or more mesodermal cell types and proliferating. As used herein mesodermal progenitors include but are not limited to, e.g. mid-primitive streak cells, paraxial mesoderm cells, lateral mesoderm cells, forelimb-forming lateral mesoderm cells, lateral plate mesoderm, splanchic mesoderm, septum transversum, pre-epicardial organ and epicardial cells. Epicardial cells are of particular interest. Such terms have well-known equivalents in the art. Both mesodermal progenitors and differentiated mesodermal cell types, e.g. cardiac pericytes, may be identified according to a variety of factors and combinations thereof. For example, identification of a particular mesodermal progenitor and differentiated mesodermal cell type is based on a particular cellular phenotype including but not limited to physical characteristics (e.g., size, shape, granularity, morphology, etc.), behavioral characteristics (e.g., movement, motility, adherence, non-adherence, etc.). Such characteristics of mesodermal progenitors and differentiated mesodermal cell types are described in, e.g., Gilbert (2006) Developmental Biology, 8th Ed. Sunderland (MA): Sinauer Associates, the disclosure of which is incorporated herein by reference in its entirety.

The terms “differentiated mesodermal cell types” and “differentiated mesodermal cells” are used interchangeably herein and refer to mesodermally derived cells that have terminally differentiated and are readily identifiable as such. Such differentiated mesodermal cell types may or may not be proliferative. As described herein, differentiated mesodermal cell types include those adult cell types and cells of adult tissues derived from mesoderm that are well-known to the ordinary skilled artisan.

The term “population”, e.g., “cell population” or “population of cells”, as used herein means a grouping (i.e., a population) of two or more cells that are separated (i.e., isolated) from other cells and/or cell groupings. For example, a 6-well culture dish can contain 6 cell populations, each population residing in an individual well. The cells of a cell population can be, but need not be, clonal derivatives of one another. A cell population can be derived from one individual cell. For example, if individual cells are each placed in a single well of a 6-well culture dish and each cell divides one time, then the dish will contain 6 cell populations. The cells of a cell population can be, but need not be, derived from more than one cell, i.e. non-clonal. The cells from which a non-clonal cell population may be derived may be related or unrelated and include but are not limited to, e.g., cells of a particular tissue, cells of a particular sample, cells of a particular lineage, cells having a particular morphological, physical, behavioral, or other characteristic, etc. A cell population can be any desired size and contain any number of cells greater than one cell. For example, a cell population can be 2 or more, 10 or more, 100 or more, 1,000 or more, 5,000 or more, 10⁴ or more, 10⁵ or more, 10⁶ or more, 10⁷ or more, 10⁸ or more, 10⁹ or more, 10¹⁰ or more, 1011 or more, 1012 or more, 1013 or more, 1014 or more, 1015 or more, 1016 or more, 1017 or more, 1018 or more, 1019 or more, or 1020 or more cells.

The terms “homogenous population”, as it relates to cell populations, refers to a cell population that is essentially pure and does not consist of a significant amount of undesired or contaminating cell types. By significant amount, in this context, is meant an amount of undesired or contaminating cell types that negatively impacts the use of the isolated desired cell population. As such, the actual amount of undesired or contaminating cells that defines a significant amount will vary and depend on the particular type of undesired or contaminating cells and/or the particular use of the desired cell type. For example, in a population of differentiated mesodermal cells used in the treatment of a subject, a significant amount of improperly differentiated contaminating cell types will be small as such cells may a high capacity to negatively impact the use of the generated desired cell population. In comparison, e.g., in a population of differentiated mesodermal cells used in the treatment of a subject, a significant amount of contaminating progenitor cells may be relatively large as such cells may have a low capacity to negatively impact the use of the generated desired cell population. In some instances, a homogenous population may refer to a highly enriched population. Levels of homogeneity will vary, as described, and may, in some instances, be greater than 60% pure, including e.g., more than 65%, more than 70%, more than 75%, more than 80%, more than 85%, more than 90%, more than 95%, more than 96%, more than 97%, more than 98%, more than 99%, more than 99.5%, more than 99.6%, more than 99.7%, more than 99.8%, and more than 99.9%.

The term “heterologous”, as it refers to a “heterologous sequence” or “heterologous nucleic acid”, means derived from a genotypically distinct entity from that of the rest of the entity to which it is being compared. For example, a polynucleotide introduced by genetic engineering techniques into a plasmid or vector derived from a different species is a heterologous polynucleotide. A promoter removed from its native coding sequence and operatively linked to a coding sequence with which it is not naturally found linked is a heterologous promoter.

Generation of mesodermal cell types from pluripotent progenitors as described herein generally involves one lineage restriction event in which cultured pluripotent progenitor cells are subjected to treatments causing the cultured cells or a population thereof to take on the features of one specific mesodermal cell type. In certain instances, lineage restriction events may be performed successively such that a first mesodermal cell type may be achieved by a first linage restriction event and the first cell type may be subjected to a second lineage restriction event to achieve a desired second mesodermal cell type.

In many instances, homogeneous populations of specific mesodermal cell-types are produced by contacting cells with various signaling pathway modulators that promote the formation of a desired cell type and various signaling pathway modulators that block the formation of other celltypes.

Pericytes are periendothelial mesenchymal cells that reside within the microvasculature, sharing a basement membrane with underlying endothelial cells. Classically described to be present on capillaries, there is considerable evidence to suggest that pericytes are ubiquitous in higher order vessels such as pre-capillary arterioles, post-capillary venules, and veins while conspicuously absent in the lymphatic vasculature. In contrast to arteriolar vSMCs, pericytes have a nearly rounded cell body with numerous finger-like projections that extend longitudinally spanning the abluminal surface of several endothelial cells. These primary processes extending along the length of the capillary give rise to secondary processes that run perpendicular to the primary processes, partially encircling the capillary with tips of secondary processes making connections with endothelial cells. In addition to forming connections with underlying capillary endothelial cells, pericytes connect with endothelial cells in neighboring capillaries with fine processes that traverse the intercapillary space cells.

Molecular markers have been suggested for identifying pericytes. Widely recognized pericyte markers include platelet-derived growth factor receptor beta (PDGFRβ), NG2 (chondroitin sulfate proteoglycan 4), CD13, alpha smooth muscle actin (αSMA), desmin, and CD146. In skeletal muscle, the expression of alkaline phosphatase by pericytes has been used to distinguish them from Pax7 (paired box protein 7) or MyoD expressing satellite cells, which are frequently in close anatomic apposition.

Cardiac pericytes are primarily derived from the epicardium, a single layer of flattened epithelial cells that surrounds the outer layer of the myocardium. During cardiac development, epicardial cells undergo epithelial to mesenchymal transition (EMT) and generate mesenchymal cells that subsequently invade the developing myocardium and give rise to cardiac fibroblasts, pericytes, and coronary vascular smooth muscle cells.

Pericytes play multiple roles in the homeostasis of skeletal and cardiac muscle, including regulation of microvascular function and angiogenesis. In addition, emerging evidence suggests a central role for pericytes in skeletal muscle formation, including modulation of angiogenesis.

Lineage restriction events as described herein may be induced by induction compositions wherein an induction composition is a composition that contains one or more induction agents useful in guiding cellular development or lineage restricting a cell along a particular lineage. Induction agents include those agents that activate or inhibit particular developmental signaling pathways that drive development. Such signaling pathways that may be activated or inhibited by induction agents include but are not limited to those signaling pathways that upon activation and inhibition generally promote mesodermal differentiation. As will be clear from the instant disclosure, whether activation or inhibition of a particular signaling pathway is necessary to generate a particular mesodermal cell type of interest will depend on a number of factors including but not limited to, e.g., the particular desired mesodermal cell type, the timing of use of the particular inductive agent and/or induction composition, the starting cell type to be induced, etc.

In some instances, an agent useful in a particular induction composition may include an activator or inhibitor of the TGF-beta (transforming growth factor β (TGF-β)) pathway. Activators and inhibitors of the TGF-beta pathway include small molecule activators, small molecule inhibitors, peptide activators, peptide inhibitors, antibodies, nucleic acid activators, nucleic acid inhibitors, and the like that activate or inhibit at least one component of the TGF-beta pathway resulting in a corresponding activation or inhibition in cellular TGF-beta signaling. Components and downstream effectors of the TGF-beta pathway include but are not limited to, e.g., 14-3-3 e (UniProtID P62258), ark (UniProtID Q6ZNA4), axin1 (UniProtID O15169), bambi (UniProtID Q13145), beta arrestin 2 (UniProtID P32121), beta catenin (UniProtID P35222), beta glycan (UniProtID Q03167), camkiia (UniProtID Q9UQM7), caveolin-1 (UniProtID Q03135), ctgf (UniProtID P29279), dab2 (UniProtID P98082), dapper2 (UniProtID Q5SW24), daxx (UniProtID Q9UER7), eif2a (UniProtID Q9BY44), elf (UniProtID Q01082), endofin (UniProtID Q7Z3T8), fkbp12 (UniProtID P62942), gadd34 (UniProtID O75807), grb2 (UniProtID P62993), itch (UniProtID Q96J02), km23-1 (UniProtID Q9NP97), nedd4-2 (UniProtID Q96PU5), ocln (UniProtID Q16625), p70s6k (UniProtID P23443), par6 (UniProtID Q9NPB6), pdk1 (UniProtID O15530), pml (UniProtID P29590), ppp1ca (UniProtID P62136), ppp2ca (UniProtID P67775), ppp2cb (UniProtID P62714), ppp2r2a (UniProtID P63151), rhoa (UniProtID P61586), sara (UniProtID 095405), she (UniProtID P29353), smad2 (UniProtID Q15796), smad3 (UniProtID P84022), smad4 (UniProtID Q13485), smad7 (UniProtID O15105), smurf1 (UniProtID Q9HCE7), smurf2 (UniProtID Q9HAU4), snon (UniProtID P12757), sos1 (UniProtID Q07889), strap (UniProtID Q9Y3F4), tab1 (UniProtID Q15750), tab2 (UniProtID Q9NYJ8), tak1 (UniProtID 043318), TGFB1 (UniProtID P01137), TGFB2 (UniProtID P61812), TGFB3 (UniProtID P10600), tgfbr1 (UniProtID P36897), tgfbr2 (UniProtID P37173), trap-1 (UniProtID O60466), wwp1 (UniProtID Q9HOMO), xiap (UniProtID P98170), yap65 (UniProtID P46937), and the like.

Activators of the TGF-beta pathway include but are not limited to, e.g., TGF-beta family ligands (e.g., TGF-beta proteins and other activators of TGF-beta receptors) and portions thereof, Activin A, TGF-beta1, TGF-beta2, TGF-beta3, IDE1/2 (IDE1 (1-[2-[(2-Carboxyphenyl)methylene]hydrazide]heptanoic acid), IDE2 (Heptanedioic acid-1-(2-cyclopentylidenehydrazide)), Nodal, and the like. In some instances, activation of the TGF-beta pathway may be achieved through repression of a TGF-beta pathway inhibitor, e.g., including but not limited to the use of an inhibitory nucleic acid targeting an inhibitor of the TGF-beta pathway or an antibody or small molecule directed to a TGF-beta pathway inhibitor.

Inhibitors of the TGF-beta pathway include but are not limited to, e.g., A-83-01 (3-(6-Methyl-2-pyridinyl)-N-phenyl-4-(4-quinolinyl)-1H-pyrazole-1-carbothioamide), D4476 (4-[4-(2,3-Dihydro-1,4-benzodioxin-6-yl)-5-(2-pyridinyl)-1 H-imidazol-2-yl]benzamide), GW 788388 (4-[4-[3-(2-Pyridinyl)-1H-pyrazol-4-yl]-2-pyridinyl]-N-(tetrahydro-2H-pyran-4-yl)-benzamide), LY 364947 (4-[3-(2-Pyridinyl)-1H-pyrazol-4-yl]-quinoline), RepSox (2-(3-(6-Methylpyridine-2-yl)-1H-pyrazol-4-yl)-1,5-naphthyridine), SB431542 (4-[4-(1,3-benzodioxol-5-yl)-5-(2-pyridinyl)-1H-imidazol-2-yl]benzamide), SB-505124 (2-[4-(1,3-Benzodioxol-5-yl)-2-(1,1-dimethylethyl)-1H-imidazol-5-yl]-6-methyl-pyridine), SB 525334 (6-[2-(1,1-Dimethylethyl)-5-(6-methyl-2-pyridinyl)-1 H-imidazol-4-yl]quinoxaline), SD208 (2-(5-Chloro-2-fluorophenyl)-4-[(4-pyridyl)amino]pteridine), ITD1 (4-[1,1′-Biphenyl]-4-yl-1,4,5,6,7,8-hexahydro-2,7,7-trimethyl-5-oxo-3-quinolinecarboxylic acid ethyl ester), DAN/Fc, antibodies to TGF-beta and TGF-beta receptors, TGF-beta inhibitory nucleic acids, and the like.

In some instances, an inducing agent useful in a particular induction composition may include an activator or inhibitor of the Wnt pathway. Activators and inhibitors of the Wnt pathway include small molecule activators, small molecule inhibitors, peptide activators, peptide inhibitors, antibodies, nucleic acid activators, nucleic acid inhibitors, and the like that activate or inhibit at least one component of the Wnt pathway resulting in a corresponding activation or inhibition in cellular Wnt signaling. Components and downstream effectors of the Wnt pathway include but are not limited to, e.g., cthrc1 (UniProtID Q96CG8), dkk1 (UniProtID O94907), fzd1 (UniProtID Q9UP38), fzd10 (UniProtID Q9ULW2), fzd2 (UniProtID Q14332), fzd4 (UniProtID Q9ULV1), fzd5 (UniProtID Q13467), fzd6 (UniProtID O60353), fzd7 (UniProtID 075084), fzd8 (UniProtID Q9H461), fzd9 (UniProtID 000144), igfbp4 (UniProtID P22692), kremen 1 (UniProtID Q96MU8), kremen 2 (UniProtID Q8NCW0), Irp5 (UniProtID O75197), Irp6 (UniProtID 075581), prr (UniProtID 075787), ror2 (UniProtID Q01974), rspo1 (UniProtID Q2MKA7), ryk (UniProtID P34925), wnt inhibitory 1 (UniProtID Q9Y5W5), wnt1 (UniProtID P04628), wnt2 (UniProtID P09544), wnt3 (UniProtID P56703), wnt3a (UniProtID P56704), wnt5a (UniProtID P41221), wnt7a (UniProtID 000755), wnt7b (UniProtID P56706), CTNNB1 (UniProtID P35222), GSK3A (UniProtID P49840), GSK3B (UniProtID P49841), TNKS1 (UniProtID 095271), TNKS2 (UniProtID Q9H2K2) and the like.

Activators of the WNT pathway include but are not limited to, e.g., CHIR99021 (6-[[2-[[4-(2,4-Dichlorophenyl)-5-(5-methyl-1H-imidazol-2-yl)-2-pyrimidinyl]amino]ethyl]amino]-3-pyridinecarbonitrile), WNT family ligands (e.g., including but not limited to Wnt-1, Wnt-2, Wnt-2b, Wnt-3a, Wnt-4, Wnt-5a, Wnt-5b, Wnt-6, Wnt-7a, Wnt-7a/b, Wnt-7b, Wnt-8a, Wnt-8b, Wnt-9a, Wnt-9b, Wnt-10a, Wnt-10b, Wnt-11, Wnt-16b, etc.), RSPO co-agonists (e.g., RSPO2), lithium chloride, TDZD8 (4-Benzyl-2-methyl-1,2,4-thiadiazolidine-3,5-dione), BIO-Acetoxime ((2' Z,3′ E)-6-Bromoindirubin-3′-acetoxime), A1070722 (1-(7-Methoxyquinolin-4-yl)-3-[6-(trifluoromethyl)pyridin-2-yl]urea), HLY78 (4-Ethyl-5,6-Dihydro-5-methyl-[1,3]dioxolo[4,5-j]phenanthridine), CID 11210285 hydrochloride (2-Amino-4-(3,4-(methylenedioxy)benzylamino)-6-(3-methoxyphenyl)pyrimidine hydrochloride), WAY-316606, (hetero) arylpyrimidines, IQ1, QS11, SB-216763, DCA, and the like. In some instances, activation of the Wnt pathway may be achieved through repression of a Wnt pathway inhibitor, e.g., including but not limited to the use of an inhibitory nucleic acid targeting an inhibitor of the Wnt pathway or an antibody or small molecule directed to a Wnt pathway inhibitor.

Inhibitors of the WNT pathway include but are not limited to, e.g., C59 (4-(2-Methyl-4-pyridinyl)-N-[4-(3-pyridinyl)phenyl]benzeneacetamide), DKK1, IWP-2 (N-(6-Methyl-2-benzothiazolyl)-2-[(3,4,6,7-tetrahydro-4-oxo-3-phenylthieno[3,2-d]pyrimidin-2-yl)thio]-acetamide), Ant1.4Br, Ant 1.4Cl, Niclosamide, apicularen, bafilomycin, XAV939 (3,5,7,8-Tetrahydro-2-[4-(trifluoromethyl)phenyl]-4H-thiopyrano[4,3-d]pyrimidin-4-one), IWR-1 (4-(1,3,3a,4,7,7a-Hexahydro-1,3-dioxo-4,7-methano-2H-isoindol-2-yl)-N-8-quinolinyl-Benzamide), NSC668036 (N-[(1, 1-Dimethylethoxy) carbonyl]-L-alanyl-(2S)-2-hydroxy-3-methylbutanoyl-L-Alanine-(1S)-1-carboxy-2-methylpropyl ester hydrate), 2,4-diamino-quinazoline, Quercetin, ICG-001 ((6S,9aS)-Hexahydro-6-[(4-hydroxyphenyl)methyl]-8-(1-naphthalenylmethyl)-4,7-dioxo-N-(phenylmethyl)-2H-pyrazino[1,2-a]pyrimidine-1 (6H)-carboxamide), PKF115-584, BML-284 (2-Amino-4-[3,4-(methylenedioxy)benzylamino]-6-(3-methoxyphenyl)pyrimidine), FH-535, iCRT-14, JW-55, JW-67, antibodies to Wnts and Wnt receptors, Wnt inhibitory nucleic acids, and the like.

In some instances, an inducing agent useful in a particular induction composition may include an activator of the FGF pathway. In some instances, an activator of the FGF pathway may also include activators of related signal transduction pathways including but not limited to, e.g., the MAPK/ERK signal transduction pathway. Activators of the FGF pathway include small molecule activators, small molecule inhibitors, peptide activators, peptide inhibitors, antibodies, nucleic acid activators, nucleic acid inhibitors, and the like that activate or inhibit at least one component of the FGF pathway resulting in a corresponding activation or inhibition in cellular FGF signaling. Components and downstream effectors of the FGF pathway include but are not limited to, e.g., akt1 (UniProtID P31749), beta-klotho (UniProtID Q86Z14), camkila (UniProtID Q9UQM7), cb1 (UniProtID P22681), cortactin (UniProtID Q14247), e-cadherin (UniProtID P12830), erk1 (UniProtID P27361), erk2 (UniProtID P28482), FGF1 (UniProtID P05230), FGF16 (UniProtID O60258), FGF17 (UniProtID O60258), FGF18 (UniProtID 076093), FGF19 (UniProtID O95750), FGF2 (UniProtID P09038), fgf23 (UniProtID Q9GZV9), FGF4 (UniProtID P08620), FGF6 (UniProtID P10767), FGF8 (UniProtID P55075), FGF9 (UniProtID P31371), fgfr1 (UniProtID P11362), fgfr2 (UniProtID P21802), fgfr2b (UniProtID P21802-18), FGFR2c (UniProtID P21802-5), FGFR3c (UniProtID P22607-1), FGFR4 (UniProtID P22455), fos (UniProtID P01100), frs2 (UniProtID Q8WU20), gab1 (UniProtID Q13480), grb2 (UniProtID P62993), hgf (UniProtID P14210), jun (UniProtID P05412), klotho (UniProtID Q9UEF7), mapk 14 (UniProtID Q16539), met (UniProtID P08581), mkp-3 (UniProtID Q16828), mmp9 (UniProtID P14780), n-cad-ctfl (UniProtID P19022), n-cad-ctf2 (UniProtID P19022), n-cadherin (UniProtID P19022), ncam (UniProtID P13591), osteocalcin (UniProtID P02818), osteopontin (UniProtID P10451), p110-alpha (UniProtID P42336), p120ctn (UniProtID O60716), p90-rsk 1 (UniProtID Q15418), pak4 (UniProtID Q8WYL5), pak4 (UniProtID 096013), pdk1 (UniProtID O15530), pik3r1 (UniProtID P27986), plcgamma1 (UniProtID P19174), pro-e-cadherin (UniProtID P12830), pro-mmp9 (UniProtID P14780), ps1 (UniProtID gamma), pyk2 (UniProtID Q14289), runx2 (UniProtID Q13950), se-cad (UniProtID P12830), secad-ntf2 (UniProtID P12830), sef (UniProtID Q8NFM7), shc (UniProtID P29353), shp2 (UniProtID Q06124), sn-cad (UniProtID P19022), sos1 (UniProtID Q07889), sprouty2 (UniProtID O43597), src (UniProtID P12931), stat1 (UniProtID P42224), stat3 (UniProtID P40763), stat5b (UniProtID P51692), syndecan-2 (UniProtID P34741), syndecan-4 (UniProtID P31431), upa (UniProtID P00749), upar (UniProtID Q03405), and the like. Activators and inhibitors of the MAPK/ERK pathway include small molecule activators, small molecule inhibitors, peptide activators, peptide inhibitors, antibodies, nucleic acid activators, nucleic acid inhibitors, and the like that activate or inhibit at least one component of the MAPK/ERK pathway resulting in a corresponding activation or inhibition in cellular MAPK/ERK signaling. Components and downstream effectors of the MAPK/ERK pathway MAPK/ERK signaling include but are not limited to, e.g., a-raf (EntrezGeneID 369), ask1 (EntrezGenelD 4217), atf2 (EntrezGenelD 1386), cebpa (EntrezGenelD 1050), c-myc (EntrezGenelD 4609), creb (EntrezGenelD 1385), elk1 (EntrezGenelD 2002), erk5 (EntrezGenelD 5598), fos (EntrezGenelD 2353), grb2 (EntrezGenelD 2885), hexokinase type iv glucokinase (EntrezGenelD 2645), ikk-alpha (EntrezGeneID 1147), ikk-beta (EntrezGenelD 3551), jnk (EntrezGenelD 5599), jun (EntrezGeneID 3725), map2k1 (EntrezGenelD 5604), map2k2 (EntrezGeneID 5605), map2k4 (EntrezGenelD 6416), map2k5 (EntrezGeneID 5607), map2k6 (EntrezGenelD 5608), map2k7 (EntrezGeneID 5609), map3k1 (EntrezGenelD 4214), map3k11 (EntrezGenelD 4296), map3k12 (EntrezGenelD 7786), map3k13 (EntrezGenelD 9175), map3k14 (EntrezGenelD 9020), map3k2 (EntrezGenelD 10746), map3k3 (EntrezGenelD 4215), map3k4 (EntrezGenelD 4216), map3k7 (EntrezGeneID 6885), map3k8 (EntrezGenelD 1326), map4k1 (EntrezGenelD 11184), map4k3 (EntrezGenelD 8491), map4k5 (EntrezGenelD 11183), mapk1 (EntrezGenelD 5594), mapk10 (EntrezGenelD 5602), mapk11 (EntrezGenelD 5600), mapk12 (EntrezGeneID 6300), mapk13 (EntrezGeneID 5603), mapk14 (EntrezGenelD 1432), mapk3 (EntrezGenelD 5595), mapk9 (EntrezGenelD 5601), max (EntrezGenelD 4149), mef2 polypeptide a (EntrezGenelD 4205), mef2 polypeptide c (EntrezGenelD 4208), mef2b (EntrezGenelD 4207), mef2 polypeptide d (EntrezGenelD 4209), mek3 (EntrezGenelD 5606), mknk2 (EntrezGenelD 2872), mnk1 (EntrezGenelD 8569), msk1 (EntrezGeneID 9252), ngf r (EntrezGenelD 4804), ngfb (EntrezGeneID 4803), nik (EntrezGenelD 9448), pak1 (EntrezGeneID 5058), pak2 (EntrezGenelD 5062), pp2a (EntrezGenelD 5528), ptprr (EntrezGenelD 5801), rac1 (EntrezGenelD 5879), raf1 (EntrezGenelD 5894), ras (EntrezGenelD 3265), rps6ka1 (EntrezGenelD 6195), shc (EntrezGenelD 6464), sos1 (EntrezGenelD 6654), sp1 (EntrezGeneID 6667), src (EntrezGenelD 6714), stat1 (EntrezGenelD 6772), stat3 (EntrezGeneID 6774), tert (EntrezGeneID 7015), and the like.

Activators of the FGF pathway and/or the MAPK/ERK pathway include but are not limited to, e.g., FGF family ligands (e.g., FGF1, FGF2, FGF-3, FGF-4, FGF-5, FGF-6, KGF/FGF-7, FGF-8, FGF-9, FGF-10, FGF-11, FGF-12, FGF-13, FGF-15, FGF-16, FGF-17, FGF-19, FGF-20, FGF-21, FGF-22, FGF-23, etc.), SUN 11602 (4-[[4-[[2-[(4-Amino-2,3,5,6-tetramethylphenyl)amino]acetyl]methylamino]-1-piperidinyl]methypenzamide), t-Butylhydroquinone, U-46619, C2 Ceramide, Lactosyl Ceramide, Angiotensin II, Baicalin, and the like. In some instances, activation of the FGF pathway and/or the MAPK/ERK pathway may be achieved through repression of the a FGF pathway and/or the MAPK/ERK pathway inhibitor, e.g., including but not limited to the use of an inhibitory nucleic acid targeting an inhibitor of the FGF pathway and/or the MAPK/ERK pathway or an antibody or small molecule directed to a FGF pathway inhibitor and/or MAPK/ERK pathway inhibitor.

In some instances, an inducing agent useful in a particular induction composition may include an activator of the BMP pathway. Activators of the BMP pathway include small molecule activators, small molecule inhibitors, peptide activators, peptide inhibitors, antibodies, nucleic acid activators, nucleic acid inhibitors, and the like that activate at least one component of the BMP pathway resulting in a corresponding activation in cellular BMP signaling. Components and downstream effectors of the BMP pathway include but are not limited to, e.g., bambi (UniProtID Q13145), bmp2 (UniProtID P12643), bmp4 (UniProtID P12644), bmp6 (UniProtID P22004), bmp7 (UniProtID P18075), bmpr1a (UniProtID P36894), bmpr1b (UniProtID 000238), bmpr2 (UniProtID Q13873), cer1 (UniProtID 095813), chrd (UniProtID Q9H2X0), chrdl1 (UniProtID Q9BU40), endofin (UniProtID Q7Z3T8), erk2 (UniProtID P28482), fetua (UniProtID P02765), fs (UniProtID P19883), gadd34 (UniProtID O75807), grem1 (UniProtID 060565), gsk3beta (UniProtID P49841), nog (UniProtID Q13253), nup214 (UniProtID P35658), ppm1a (UniProtID P35813), ppp1ca (UniProtID P62136), rgma (UniProtID Q96B86), rgmb (UniProtID Q6NW40), rgmc (UniProtID Q6ZVN8), scp1 (UniProtID Q9GZU7), scp2 (UniProtID 014595), scp3 (UniProtID 015194), ski (UniProtID P12755), smad1 (UniProtID Q15797), smad4 (UniProtID Q13485), smad5 (UniProtID Q99717), smad6 (UniProtID O43541), smad7 (UniProtID 015105), smad8a (UniProtID 015198), smurf1 (UniProtID Q9HCE7), smurf2 (UniProtID Q9HAU4), tab1 (UniProtID Q15750), tab2 (UniProtID Q9NYJ8), tak1 (UniProtID O43318), usag1 (UniProtID Q6X4U4), xiap (UniProtID P98170), and the like.

Activators of the BMP pathway include but are not limited to, e.g., BMP family ligands e.g., BMP2, BMP4, BMP7, etc., Alantolactone, FK506, isoliquiritigenin, 4′-hydroxychalcone, and the like. In some instances, activation of the BMP pathway may be achieved through repression of a BMP pathway inhibitor, e.g., including but not limited to the use of an inhibitory nucleic acid targeting an inhibitor of the BMP pathway or an antibody or small molecule directed to a BMP pathway inhibitor.

In some instances, an inducing agent useful in a particular induction composition may include an activator of the retinoic acid signaling pathway. Activators of the retinoic acid signaling pathway include small molecule activators, small molecule inhibitors, peptide activators, peptide inhibitors, antibodies, nucleic acid activators, nucleic acid inhibitors, and the like that activate at least one component of the retinoic acid signaling pathway resulting in a corresponding activation in cellular retinoic acid signaling. Components and downstream effectors of the retinoic acid signaling pathway include but are not limited to, e.g., CRABP (e.g., Accession: NP_004369), TRAIL (e.g., Accession: NP_003801), TRAILR1 (e.g., Accession: NP_003835), TRAILR2 (e.g., Accession: NP_003833), DAP3 (e.g., Accession: NP_001186780), FADD (e.g., Accession: CAG33019), FLIP (e.g., Accession: NP_001294972), Caspase 8 (e.g., Accession: AAD24962), BID (e.g., Accession: NP_001304162), tBID (e.g., Accession: P55957), APAF1 (e.g., Accession: ABQ59028), Caspase 9 (e.g., Accession: P55211), PARPs (e.g., Accession: AAH14206), RAR (e.g., Accession: NP_001138773 and components thereof e.g., AF2 domain, AF1 domain, DBD domain, and the like. Activators of the retinoic acid signaling include but are not limited to e.g., Tretinoin, Retinol palmitate, Etretinate, Isotretinoin, Adapalene, Tazarotene, Tamibarotene, Retinol acetate, Acitretin, Alitretinoin, Bexarotene, Isotretinoin anisatil, Motretinide, Vitamin A, Retinol propionate, and the like. In some instances, useful modulators of the retinoic acid signaling pathway include retinoid agonist, including but not limited to e.g., all-trans retinoic acid, TTNPB, AM580 and the like. In some instances, an inducing agent useful in a particular induction composition may include an activator of the Hedgehog pathway. Activators of the Hedgehog pathway include small molecule activators, small molecule inhibitors, peptide activators, peptide inhibitors, antibodies, nucleic acid activators, nucleic acid inhibitors, and the like that activate at least one component of the Hedgehog pathway resulting in a corresponding activation or inhibition in cellular Hedgehog signaling. Components and downstream effectors of the Hedgehog pathway include but are not limited to, e.g., akt1 (UniProtID P31749), beta arrestin2 (UniProtID P32121), boc (UniProtID Q9BWV1), cdo (UniProtID Q4KMG0), dhh (UniProtID O43323), gas1 (UniProtID P54826), gli2 (UniProtID P10070), grk2 (UniProtID P25098), hhat (UniProtID Q5VTY9), hhip (UniProtID Q96QV1), ihh (UniProtID Q14623), Irpap1 (UniProtID P30533), megalin (UniProtID P98164), p110-alpha (UniProtID P42336), pik3r1 (UniProtID P27986), ptch1 (UniProtID Q13635), ptch2 (UniProtID Q9Y6C5), pthrp (UniProtID P12272), shh (UniProtID Q15465), sil (UniProtID Q15468), smo (UniProtID Q99835), tgf-beta2 (UniProtID P61812), and the like.

In some instances, an inducing agent useful in a particular induction composition may include an inhibitor of the PI3K pathway. Inhibitors of the PI3K pathway include small molecule activators, small molecule inhibitors, peptide activators, peptide inhibitors, antibodies, nucleic acid activators, nucleic acid inhibitors, and the like that inhibit at least one component of the PI3K pathway resulting in a corresponding inhibition in cellular PI3K signaling. Components and downstream effectors of the PI3K pathway include but are not limited to, e.g., arap3 (UniProtID Q8WWN8), arf1 (UniProtID P84077), arf5 (UniProtID P84085), arf6 (UniProtID P62330), arno (UniProtID Q99418), bam32 (UniProtID Q9UN19), blk (UniProtID P51451), bink (UniProtID Q8WV28), btk (UniProtID Q06187), centa1 (UniProtID O75689), cytohesin-1 (UniProtID Q15438), fgr (UniProtID P09769), foxo3a (UniProtID O43524), fyn (UniProtID P06241), grp1 (UniProtID O43739), hck (UniProtID P08631), h-ras isoform 1 (UniProtID P01112), h-ras isoform 2 (UniProtID P01112), hsp90 (UniProtID P07900), itk (UniProtID Q08881), k-ras isoform 2a (UniProtID P01116-1), k-ras isoform 2b (UniProtID P01116-2), lat (UniProtID 043561-2), Ick (UniProtID P06239), lyn (UniProtID P07948), n-ras (UniProtID P01111), p101 (UniProtID Q8WYR1), p110-alpha (UniProtID P42336), p110-beta (UniProtID P42338), p110D (UniProtID O00329), p55-gamma (UniProtID Q92569), p84 (UniProtID Q5UE93), p85-beta (UniProtID 000459), pdk1 (UniProtID O15530), PI3Kgamma (UniProtID P48736), PIK3R1 (UniProtID P27986), plcgamma1 (UniProtID P19174), plcgamma2 (UniProtID P16885), pten (UniProtID P60484), rac1 (UniProtID P63000), rap1a (UniProtID P62834), rhoa (UniProtID P61586), sgk1 (UniProtID 000141), ship (UniProtID O00145), ship2 (UniProtID 015357), src (UniProtID P12931), syk (UniProtID P43405), tapp1 (UniProtID Q9HB19), tapp2 (UniProtID Q9HB21), yes (UniProtID P07947), zap-70 (UniProtID P43403), and the like.

Inhibitors of the PI3K pathway include but are not limited to, e.g., AS 252424 (5-[[5-(4-Fluoro-2-hydroxyphenyl)-2-furanyl]methylene]-2,4-thiazolidinedione), AS 605240 (5-(6-Quinoxalinylmethylene)-2,4-thiazolidine-2,4-dione), AZD 6482 ((-)-2-[[(1R)-1-[7-Methyl-2-(4-morpholinyl)-4-oxo-4H-pyrido[1,2-a]pyrimidin-9-yl]ethyl]amino]benzoic acid), BAG 956 (α,α,-Dimethyl-4-[2-methyl-8-[2-(3-pyridinyl) ethynyl]-1H-imidazo[4,5-c]quinolin-1-yl]-benzeneacetonitrile), CZC 24832 (5-(2-Amino-8-fluoro[1,2,4]triazolo[1,5-a]pyridin-6-yl)-N-(1,1-dimethylethyl)-3-pyridinesulfonamide), GSK 1059615 (5-[[4-(4-Pyridinyl)-6-quinolinyl]methylene]-2,4-thiazolidenedione), KU 0060648 (4-Ethyl-N-[4-[2-(4-morpholinyl)-4-oxo-4H-1-benzopyran-8-yl]-1-dibenzothienyl]-1-piperazineacetamide), LY 294002 hydrochloride (2-(4-Morpholinyl)-8-phenyl-4H-1-benzopyran-4-one hydrochloride), 3-Methyladenine (3-Methyl-3H-purin-6-amine), PF 04691502 (2-Amino-8-[trans-4-(2-hydroxyethoxy)cyclohexyl]-6-(6-methoxy-3-pyridinyl)-4-methyl-pyrido[2,3-d]pyrimidin-7 (8H)-one), PF 05212384 (N-[4-[[4-(Dimethylamino)-1-piperidinyl]carbonyl]phenyl]-N′-[4-(4,6-di-4-morpholinyl-1,3,5-triazin-2-yl)phenyl]urea), PI 10³ hydrochloride (3-[4-(4-Morpholinylpyrido[3′,2′: 4,5]furo[3,2-d]pyrimidin-2-yl]phenol hydrochloride), PI 828 (2-(4-Morpholinyl)-8-(4-aminophenyl)-4H-1-benzopyran-4-one), PP 121 (1-Cyclopentyl-3-(1H-pyrrolo[2,3-b]pyridin-5-yl)-1H-pyrazolo[3,4-d]pyrimidin-4-amine), Quercetin, TG 100713 (3-(2,4-Diamino-6-pteridinyl)-phenol), Wortmannin, PIK90, GDC-0941, antibodies to PI3K and PI3K receptors, PI3K inhibitory nucleic acids, and the like.

In some instances, an inducing agent useful in a particular induction composition may include an activator of the PDGF pathway. Activators of the PDGF pathway include small molecule activators, small molecule inhibitors, peptide activators, peptide inhibitors, antibodies, nucleic acid activators, nucleic acid inhibitors, and the like that activate or inhibit at least one component of the PDGF pathway resulting in a corresponding activation or inhibition in cellular PDGF signaling. Components and downstream effectors of the PDGF pathway include but are not limited to, e.g., 14-3-3 e (UniProtID P62258), abi1 (UniProtID Q8IZPO), acta2 (UniProtID P62736), afadin (UniProtID P55196), alpha actinin 4 (UniProtID O43707), alphav integrin (UniProtID P06756), arap1 (UniProtID Q96P48), arp2 (UniProtID P61160), arp3 (UniProtID P61158), arpc1b (UniProtID 015143), arpc2 (UniProtID O15144), arpc3 (UniProtID O15145), arpc4 (UniProtID P59998), arpc5 (UniProtID O15511), beta3 integrin (UniProtID P05106), blk (UniProtID P51451), braf (UniProtID P15056), c3g (UniProtID Q13905), c-abl (UniProtID P00519), caveolin-1 (UniProtID Q03135), caveolin-3 (UniProtID P56539), cb1 (UniProtID P22681), ck2a1 (UniProtID P68400), cortactin (UniProtID Q14247), crk (UniProtID P46108), crk1 (UniProtID P46109), csk (UniProtID P41240), dep1 (UniProtID Q12913), dock4 (UniProtID Q8N110), dynamin 2 (UniProtID P50570), elk1 (UniProtID P19419), eps8 (UniProtID Q12929), erk1 (UniProtID P27361), erk2 (UniProtID P28482), fgr (UniProtID P09769), fos (UniProtID P01100), fyn (UniProtID P06241), gab1 (UniProtID Q13480), grb10 (UniProtID Q13322), grb2 (UniProtID P62993), hck (UniProtID P08631), h-ras isoform 1 (UniProtID P01112), h-ras isoform 2 (UniProtID P01112), hspc300 (UniProtID Q8WUW1), ifn-gamma (UniProtID P01579), idgap1 (UniProtID P46940), irsp53 (UniProtID Q9UQB8), jak1 (UniProtID P23458), jak2 (UniProtID O60674), jnk1 (UniProtID P45983), jnk2 (UniProtID P45984), jnk3 (UniProtID P53779), jun (UniProtID P05412), jund (UniProtID P17535), k-ras isoform 2a (UniProtID P01116-1), k-ras isoform 2b (UniProtID P01116-2), ksr (UniProtID Q81VT5), Ick (UniProtID P06239), Irp1 (UniProtID Q07954), lyn (UniProtID P07948), mek1 (UniProtID Q02750), mek2 (UniProtID P36507), mkk4 (UniProtID P45985), mkk7 (UniProtID O14733), myc (UniProtID P01106), myocardin (UniProtID Q81ZQ8), nap1 (UniProtID Q9Y2A7), nck1 (UniProtID P16333), nck2 (UniProtID O43639), nherf1 (UniProtID O14745), nherf2 (UniProtID Q15599), n-ras (UniProtID P01111), n-wasp (UniProtID O00401), p101 (UniProtID Q8WYR1), p110-alpha (UniProtID P42336), p110-beta (UniProtID P42338), p110D (UniProtID O00329), p130 cas (UniProtID P56945), p190rhogap (UniProtID Q9NRY4), p52 shc (UniProtID P29353-2), p55-gamma (UniProtID Q92569), p62dok (UniProtID Q99704), p84 (UniProtID Q5UE93), p85-beta (UniProtID 000459), pag1 (UniProtID Q9NWQ8), pak1 (UniProtID Q13153), pdgfa (UniProtID P04085), pdgfb (UniProtID P01127), pdgfc (UniProtID Q9NRA1), pdgfd (UniProtID Q9GZPO), pdgfra (UniProtID P16234), pdgfrb (UniProtID P09619), PI3Kgamma (UniProtID P48736), pik3r1 (UniProtID P27986), pin1 (UniProtID Q13526), pkc alpha (UniProtID P17252), pkc delta (UniProtID Q05655), pkc epsilon (UniProtID Q02156), pkr (UniProtID P19525), p1a2g4a (UniProtID P47712), plcgamma1 (UniProtID P19174), ppp2ca (UniProtID P67775), ppp2r1a (UniProtID P30153), ppp2r2b (UniProtID Q00005), pten (UniProtID P60484), ptp1b (UniProtID P18031), rab4a (UniProtID P20338), rab5 (UniProtID P20339), rac1 (UniProtID P63000), raf1 (UniProtID P04049), rap1a (UniProtID P62834), rap1b (UniProtID P61224), rasgap (UniProtID P20936), rhoa (UniProtID P61586), rhogdi (UniProtID P52565), rntre (UniProtID Q92738), rsk2 (UniProtID P51812), s1p1 (UniProtID P21453), shb (UniProtID Q15464), shc (UniProtID P29353), shf (UniProtID Q7M4L6), shp2 (UniProtID Q06124), slap (UniProtID Q13239), sm22 (UniProtID Q01995), sos1 (UniProtID Q07889), spa-1 (UniProtID Q96FS4), sphk1 (UniProtID Q9NYA1), sra1 (UniProtID Q96F07), src (UniProtID P12931), srf (UniProtID P11831), stat1 (UniProtID P42224), stat3 (UniProtID P40763), STATSA (UniProtID P42229), STATSB (UniProtID P51692), tcptp p45 (UniProtID P17706-1), vav2 (UniProtID P52735), wave2 (UniProtID Q9Y6W5), yes (UniProtID P07947), ywhab (UniProtID P31946), ywhag (UniProtID P61981), ywhah (UniProtID Q04917), ywhaq (UniProtID P27348), ywhas (UniProtID P31947), ywhaz (UniProtID P63104), and the like.

Activators of the PDGF pathway include but are not limited to, e.g., PDGF family ligands (e.g., PDGF, PDGF A, PDGF B, PDGF C, PDGF D, etc.) and fragments thereof and/or dimers thereof (e.g., PDGF-AA, PDGF-BB, PDGF-CC, PDGF-DD, PDGF-AB, etc.), and the like. In some instances, activation of the PDGF pathway may be achieved through repression of a PDGF pathway inhibitor, e.g., including but not limited to the use of an inhibitory nucleic acid targeting an inhibitor of the PDGF pathway or an antibody or small molecule directed to a PDGF pathway inhibitor.

In some instances, induction, or pathway modulating agents, as described above and including pathway activators and pathway inhibitors include, e.g., those that are commercially available, e.g., from such suppliers such as Tocris Bioscience (Bristol, UK), Sigma-Aldrich (St. Louis, Mo.), PeproTech (Thermo Fisher Scientific, MA), Santa Cruz Biotechnology (Santa Cruz, Calif.), and the like.

Pluripotent progenitors and derivatives thereof may be contacted with induction agents by any convenient means. Generally, an induction agent is added to culture media, as described herein, within which cells of the instant disclosure are grown or maintained, such that the induction agent is present, in contact with the cells, at an effective concentration to produce the desired effect, e.g., induce a desired lineage restriction event. In other instances, e.g., where the existing culture media is not compatible with a particular induction agent, the culture media in which the cells are being grown is replaced with fresh culture media containing the particular induction agent present in the fresh media at an effective concentration to produce the desired effect. In instances where fresh or specific culture media is provided with a particular induction agent the culture agent may, in some instances, be specifically formulated for the particular induction agent, e.g., containing one or more specific additional reagents to, e.g., aid in the delivery of the induction agent, aid in the solubility of the induction agent, aid in the stability of the induction agent, etc.

The effective concentration of a particular induction agent will vary and will depend on the agent. In addition, in some instances, the effective concentration may also depend on the cells being induced, the culture condition of the cells, other induction agents co-present in the culture media, etc. As such, the effective concentration of induction agents will vary and may range from 1 ng/ml to 10 μg/mL or more, including but not limited to, e.g., 1 ng/ml, 2 ng/ml, 3 ng/ml, 4 ng/ml, 5 ng/mL, 6 ng/ml, 7 ng/ml, 8 ng/mL, 9 ng/ml, 10 ng/ml, 11 ng/ml, 12 ng/ml, 13 ng/ml, 14 ng/ml, 15 ng/mL, 16 ng/ml, 17 ng/ml, 18 ng/ml, 19 ng/mL, 20 ng/ml, 21 ng/ml, 22 ng/ml, 23 ng/ml, 24 ng/mL, 25 ng/ml, 26 ng/ml, 27 ng/ml, 28 ng/mL, 29 ng/mL, 30 ng/ml, 31 ng/ml, 32 ng/ml, 33 ng/ml, 34 ng/mL, 35 ng/ml, 36 ng/ml, 37 ng/mL, 38 ng/mL, 39 ng/ml, 40 ng/ml, 41 ng/ml, 42 ng/ml, 43 ng/mL, 44 ng/ml, 45 ng/ml, 46 ng/ml, 47 ng/ml, 48 ng/ml, 49 ng/ml, 50 ng/ml, 1-5 ng/ml, 1-10 ng/mL, 1-20 ng/ml, 1-30 ng/ml, 1-40 ng/ml, 1-50 ng/ml, 5-10 ng/ml, 5-20 ng/ml, 10-20 ng/ml, 10-30 ng/ml, 10-40 ng/ml, 10-50 ng/ml, 20-30 ng/ml, 20-40 ng/ml, 20-50 ng/ml, 30-40 ng/ml, 30-50 ng/ml, 40-50 ng/ml, 1-100 ng/ml, 50-100 ng/ml, 60-100 ng/ml, 70-100 ng/ml, 80-100 ng/mL, 90-100 ng/ml, 10-100 ng/ml, 50-200 ng/ml, 100-200 ng/ml, 50-300 ng/ml, 100-300 ng/ml, 200-300 ng/ml, 50-400 ng/ml, 100-400 ng/ml, 200-400 ng/ml, 300-400 ng/mL, 50-500 ng/ml, 100-500 ng/ml, 200-500 ng/ml, 300-500 ng/ml, 400 to 500 ng/ml, 0.001-1 μg/mL, 0.001-2 μg/mL, 0.001-3 μg/mL, 0.001-4 μg/mL, 0.001-5 μg/mL, 0.001-6 μg/mL, 0.001-7 μ g/mL, 0.001-8 μ g/mL, 0.001- 9 μ g/mL, 0.001-10 μ g/mL, 0.01-1 μ g/mL, 0.01-2 μ g/mL, 0.01-3 ug/mL, 0.01-4 μg/mL, 0.01-5 μg/mL, 0.01-6 μg/mL, 0.01-7 μg/mL, 0.01-8 μg/mL, 0.01-9 ug/mL, 0.01-10 μg/mL, 0.1-1 μg/mL, 0.1-2 μg/mL, 0.1-3 μg/mL, 0.1-4 μg/mL, 0.1-5 μg/mL, 0.1-6 μg/mL, 0.1-7 μg/mL, 0.1-8 μg/mL, 0.1-9 μg/mL, 0.1-10 μg/mL, 0.5-1 μg/mL, 0.5-2 μg/mL, 0.5-3 μg/mL, 0.5-4 μg/mL, 0.5-5 g/mL, 0.5-6 μg/mL, 0.5-7 μg/mL, 0.5-8 μg/mL, 0.5-9 μg/mL, 0.5-10 μg/mL, and the like.

In some instances, the effective concentration of an induction agent in solution, e.g., cell culture media, may range from 1 nM to 100 UM or more, including but not limited to, e.g., 1 nM, 2 nM, 3 nM, 4 nM, 5 nM, 6 nM, 7 nM, 8 nM, 9 nM, 10 nM, 11 nM, 12 nM, 13 nM, 14 nM, 15 nM, 16 nM, 17 nM, 18 nM, 19 nM, 20 nM, 21 nM, 22 nM, 23 nM, 24 nM, 25 nM, 26 nM, 27 nM, 28 nM, 29 nM, 30 nM, 31 nM, 32 nM, 33 nM, 34 nM, 35 nM, 36 nM, 37 nM, 38 nM, 39 nM, 40 nM, 41 nM, 42 nM, 43 nM, 44 nM, 45 nM, 46 nM, 47 nM, 48 nM, 49 nM, 50 nM, 1-2 nM, 1-3 nM, 1-4 nM, 1-5 nM, 1-6 nM, 1-7 nM, 1-8 nM, 1-9 nM, 1-10 nM, 1.5 nM, 1.5-2 nM, 1.5-3 nM, 1.5-4 nM, 1.5-5 nM, 1.5-6 nM, 1.5-7 nM, 1.5-8 nM, 1.5-9 nM, 1.5-10 nM, 2-3 nM, 2-4 nM, 2-5 nM, 2-6 nM, 2-7 nM, 2-8 nM, 2-9 nM, 2-10 nM, 3-4 nM, 3-5 nM, 3-6 nM, 3-7 nM, 3-8 nM, 3-9 nM, 3-10 nM, 4-5 nM, 4-6 nM, 4-7 nM, 4-8 nM, 4-9 nM, 4-10 nM, 5-6 nM, 5-7 nM, 5-8 nM, 5-9 nM, 5-10 nM, 6-7 nM, 6-8 nM, 6-9 nM, 6-10 nM, 7-8 nM, 7-9 nM, 7-10 nM, 8-9 nM, 8-10 nM, 9-10 nM, 5-15 nM, 5-20 nM, 5-25 nM, 5-30 nM, 5-35 nM, 5-40 nM, 5-45 nM, 5-50 nM, 10-15 nM, 10-20 nM, 10-25 nM, 10-30 nM, 10-35 nM, 10-40 nM, 10-50 nM, 15-20 nM, 15-25 nM, 15-30 nM, 15-35 nM, 15-40 nM, 15-45 nM, 15-50 nM, 20-25 nM, 20-30 nM, 20-35 nM, 20-40 nM, 20-45 nM, 20-50 nM, 25-30 nM, 25-35 nM, 25-40 nM, 25-45 nM, 25-50 nM, 30-35 nM, 30-40 nM, 30-45 nM, 30-50 nM, 35-40 nM, 35-45 nM, 35-50 nM, 40-45 nM, 40-50 nM, 45-50 nM, 10-100 nM, 20-100 nM, 30-100 nM, 40-100 nM, 50-100 nM, 60-100 nM, 70-100 nM, 80-100 nM, 90-100 nM, 50-150 nM, 50-200 nM, 50-250 nM, 50-300 nM, 50-350 nM, 50-400 nM, 50-450 nM, 50-500 nM, 10-150 nM, 10-200 nM, 10-250 nM, 10-300 nM, 10-350 nM, 10-400 nM, 10-450 nM, 10-500 nM, 100-150 nM, 100-200 nM, 100-250 nM, 100-300 nM, 100-350 nM, 100-400 nM, 100-450 nM, 100-500 nM, 200-500 nM, 300-500 nM, 400-500 nM, 100 nM, 150 nM, 200 nM, 250 nM, 300 nM, 350 nM, 400 nM, 450 nM, 500 nM, 550 nM, 600 nM, 650 nM, 700 nM, 750 nM, 800 nM, 850 nM, 900 nM, 950 nM, 200-400 nM, 300-500 nM, 400-600 nM, 500-700 nM, 600-800 nM, 700-900 nM, 800 nM to 1 μM, 0.5-1 μM, 0.5-1.5 μM, 0.5-2 μM, 0.5-2.5 μM, 0.5-3 μM, 0.5-3.5 μM, 0.5-4 μM, 0.5-4.5 μM, 0.5-5 μM, 1 μM, 2 μM, 3 μM, 4 μM, 5 μM, 6 μM, 7 μM, 8 μM, 9 M, 10 μM, 11 μM, 12 μM, 13 μM, 14 μM, 15 μM, 16 μM, 17 μM, 18 μM, 19 μM, 20 μM, 21 μM, 22 μM, 23 μM, 24 μM, 25 μM, 26 μM, 27 μM, 28 μM, 29 μM, 30 μM, 31 μM, 32 μM, 33 μM, 34 μM, 35 μM, 36 μM, 37 μM, 38 μM, 39 μM, 40 μM, 41 μM, 42 μM, 43 μM, 44 μM, 45 μM, 46 μM, 47 μM, 48 μM, 49 μM, 50 μM, 1-2 μM, 1-3 μM, 1-4 μM, 1-5 μM, 1-6 μM, 1-7 μM, 1-8 μM, 1-9 μM, 1-10 μM, 1.5 μM, 1.5-2 μM, 1.5-3 μM, 1.5-4 μM, 1.5-5μ, 1.5-6μ, 1.5-7μ, 1.5-8μ, 1.5-9μ, 1.5-10μ, 2-3μ, 2-4μ, 2-5μ, 2-6 μM, 2-7 μM, 2-8 μM, 2-9 μM, 2-10 μM, 3-4 μM, 3-5 μM, 3-6 μM, 3-7 μM, 3-8 μM, 3-9 μM, 3-10μ, 4-5μ, 4-6μ, 4-7μ, 4-8μ, 4-9μ, 4-10μ, 5-6μ, 5-7μ, 5-8μ, 5-9μ, 5-10 μM, 6-7 μM, 6-8 μM, 6-9 μM, 6-10 μM, 7-8 μM, 7-9 μM, 7-10 μM, 8-9 μM, 8-10 μM, 9-10 μM, 5-15 μM, 5-20 μM, 5-25 M, 5-30 M, 5-35 M, 5-40 MM, 5-45 MM, 5-50 μM, 10-15 μM, 10-20 μM, 10-25 μM, 10-30 μM, 10-35 μM, 10-40 μM, 10-50 μM, 15-20 μM, 15-25 μM, 15-30 μM, 15-35 μM, 15-40 μM, 15-45 μM, 15-50 μM, 20-25 μM, 20-30 μM, 20-35 μM, 20-40 μM, 20-45 M, 20-50 μM, 25-30 μM, 25-35 M, 25-40 μM, 25-45 M, 25-50 μM, 30-35 μM, 30-40 μM, 30-45 M, 30-50 μM, 35-40 μM, 35-45 μM, 35-50 μM, 40-45 μM, 40-50 μM, 45-50 μM, 10-100 μM, 20-100 μM, 30-100 μM, 40-100 μM, 50-100 μM, 60-100 μM, 70-100 μM, 80-100 μM, 90-100 μM, and the like.

In some instances, the effective concentration of an induction agent will be below a critical concentration such that the induction produces the desired effect without undesirable effects. As used herein, the term “critical concentration” refers to a concentration of induction agent above which undesirable effects are produced. Undesirable effects that may be the result of a concentration exceeding the critical concentration include but are not limited to, e.g., off-target effects (off-target activation of signaling, off-target inhibition of signaling), reduction or loss of function (e.g., loss of desired activator function, loss of desired inhibitor function) reduction of cell viability, increase in cell mortality, lineage restriction towards an undesired cell type, differentiation into an undesired cell type, loss of expression of a particular desired marker, etc. Whether a particular induction agent will have a critical concentration and what the critical concentrations of those agents having a critical concentration are will depend on the agent and the specific conditions in which the agent is used.

In some instances, cells of the instant disclosure may be contacted with multiple induction agents and/or multiple induction compositions in order achieve a desired mesodermal cell type and terminally differentiated derivative thereof. In some instances, a particular induction composition will contain two or more induction agents such that a particular cell culture is simultaneously contacted with multiple induction agents. In some instances, a particular series of induction compositions may be used, one at a time, in generating a desired mesodermal cell type such that a particular cell culture is successively contacted with multiple induction agents.

The duration of contact of a particular induction composition with a particular cell type will vary and will depend, e.g., on the desired mesodermal cell type, the cell type being induced, and the components of the induction composition. In some instances, a particular induction composition may be introduced for different exposure times depending on the context of use, e.g., cell type X may be contacted with induction composition Y for time Z whereas cell type A may be contacted with induction composition Y for time B, wherein cell type X is different than cell type A and time Z is different than time B. As such, the time cells are contacted with a particular induction composition may vary, e.g., when being used on different cells, when being used to generate different cells, or when being used at different steps of a differentiation process.

The duration of contact of a particular induction composition with a particular cell type, in some instances, may be referred to as the “exposure time” and exposure times may range from a day to weeks or more, including but not limited to e.g., 1 day, 1.5 days, 2 days, 2.5 days, 3 days, 3.5 days, 4 days, 4.5 days, 5 days, 5.5 days, 6 days, 6.5 days, 7 days, 7.5 days, 8 days, 8.5 days, 9 days, 9.5 days, 10 days, 11 days, 12, days, 13, days, 14 days, 15, days, etc. As used herein, exposure times are, in some instances, referred as consisting essentially of, e.g., 24 hours, indicating that the exposure time may be longer or shorter than that specified including those exposure times that are longer or shorter but do not materially affect the basic outcome of the particular exposure. As such, in some instances where a particular exposure is more time sensitive such that under or over exposure, e.g., of more or less than 1 hour, materially affects the outcome of the exposure, a time period consisting essentially of, e.g., 24 hours, will be interpreted to refer to a time period ranging from about 23 hours to about 25 hours. In some other instances where a particular exposure is less time sensitive such that under or over exposure, e.g., of more than 12 hours, does not materially affect the outcome of the exposure, a time period consisting essentially of, e.g., 24 hours will mean a time period ranging from about 12 hours or less to about 36 hours or more. In some instances, depending on the context, an exposure period consisting essentially of 24 hours may refer to an exposure time of 22-26 hours, 21-27 hours, 20-28 hours, 19-29 hours, 18-30 hours, etc.

In some instances, time periods of exposure may be pre-determined such that cells are contacted with an induction composition according to a schedule set forth prior to the contacting. In some instances, the time period of exposure, whether pre-determined or otherwise, may be modulated according to some feature or characteristic of the cells and/or cell culture, including but not limited to, e.g., cell morphology, cell viability, cell appearance, cellular behaviors, cell number, culture confluence, marker expression, etc.

Methods of modification of cells, including modification of pluripotent cells and modification of mesodermal cell types are well-known in the art and include but are not limited to e.g., genetic modification (e.g., through deletion mutagenesis, through substitution mutagenesis), through insertional mutagenesis (e.g., through the introduction of heterologous nucleic acid into the pluripotent cell, etc.), non-mutagenic genetic modification (e.g., the non-mutagenic insertion of heterologous nucleic acid, etc.), epigenetic modification (e.g., through the treatment with one or more specific or general epigenetic modifying agents (e.g., methylation inhibitors, methylation activators, demethylases, etc.), other modifications (e.g., non-genetic labeling, etc.).

Modifications of cells may be transient or stable. In some instances, a modification of a particular pluripotent cell or mesodermal progenitor cell may be stable such that the modification persists through derivation of a desired mesodermal cell type from the pluripotent cell or progenitor cell as described herein. In some instances, stable modifications may persist through introduction of a mesodermally derived cell type into a host. In some instances, stable modifications may persist through proliferation of the cell such that all progenitors of a particular modified cell also contain the subject modification. In some instances, a modification of a particular pluripotent cell or progenitor cell may be transient such that the modification is lost after derivation of a mesodermal cell type of interest from the transiently modified pluripotent cell. In certain instances, transient modifications may persist through one or more rounds of proliferation of the modified cell such that some but not all of the progeny of the modified cell contain the subject modification. In some instances, a transient modification will not persist during proliferation such that none of the progeny of a modified cell will contain the subject modification. In some instances, a transiently modified cell may be configured such that the modification persists through certain aspects of derivation of the cell type of interest, e.g., through derivation of a particular mesodermal cell type of interest, but is lost prior to introduction of the derived cell into a host.

### Generation of Epicardial Cells

In some embodiments, a stepwise method of producing a population of epicardium cells is provided. A population of pluripotent stem cells is contacted in culture with mid-primitive streak (MPS) induction media for a period of about 24 hours to induce a population of MPS cells. The mid-primitive streak induction medium may comprise an effective dose of a TGF-beta pathway activator; a Wnt pathway activator; an FGF pathway activator; a BMP pathway activator; and a PI3K pathway inhibitor.

The population of MPS cells is contacted with lateral plate mesoderm (LPM) induction medium to induce a population of LPM cells for a period of about 24 hours. The lateral plate mesoderm induction medium comprises an effective dose of a TGF-beta pathway inhibitor; a Wnt pathway inhibitor; and a BMP pathway activator.

The population of LPM cells in contacted with splanchnic mesoderm (SM) induction medium for a period of about 48 hours to induce a population of SM cells. Splanchic mesoderm induction medium comprises an effective dose of a TGF-beta pathway inhibitor; a Wnt pathway inhibitor; a BMP pathway activator; an FGF pathway activator; and a retinoic acid pathway activator.

The population of SM cells is contacted with septum transversum (ST) induction medium for a period of about 72 hours to induce a population of ST cells. The septum transversum induction medium comprises an effective dose of a BMP pathway activator; and a retinoic acid pathway activator.

The population of ST cells is contacted with proepicardium organ (PEO) induction medium for a period of from 1-3 days to induce a population of PEO cells. The proepicardium organ induction medium comprises an effective dose of a retinoic acid pathway activator; and a TGF-beta pathway inhibitor in LaSR medium or equivalent.

The population of PEO cells is contacted with epicardium (EPI) induction medium for a period of from 1-3 days to induce a population of EPI cells. The epicardium induction medium comprises an effective dose of a TGF-beta pathway inhibitor in LaSR medium or equivalent.

The population of epicardial cells is optionally contacted with pericyte induction medium for a period of from 6-12 days to induce a population of cardiac pericytes. The pericyte induction medium comprises an effective dose of a PDGF pathway activator in pericyte medium.

### Screening

Aspects of the instant disclosure include method of screening pharmacological agents using mesodermal cell types, particularly epicardial cells and cardiac pericytes, derived according to the methods described herein. In some instances, a plurality of cell populations derived according to the methods as described herein are contacted with a plurality of pharmacological agents in order to screen for agents producing a cellular response of interest. A cellular response of interest may be any cellular response including but not limited to, e.g., cell death, cell survival, cell self-renewal, proliferation, differentiation, expression of one or more markers, loss of expression of one or more markers, change in morphology, change in cellular physiology, cellular engraftment, change in cell motility, change in cell migration, production of a particular cellular component, cease of production of a particular cellular component, change in metabolic output, response to stress, and the like.

Screening pharmacological agents using cells described herein may be performed in vitro, e.g., in a tissue culture chamber, on a slide, etc., or may be performed in vivo, e.g., in an animal host, etc. Cells used in such screening assays may be genetically altered or may an unaltered cell. In some instances, cells generated according to the methods as described herein are used in multiplexed in vitro pharmacological screening. Methods for evaluating cellular responses during in vitro screening are well-known in the art and include but are not limited to, e.g., microscopic methods (e.g., light microscopy, electron microscopy, etc.), expression assays, enzymatic assays, cytological assays (e.g., cellular staining), genomics, transcriptomics, metabolomics, and the like.

In some instances, cells generated according to the methods as described herein are introduced into a host animal and the host animal may be administered a pharmacological agent in order to screen for a response from the introduced cells. In some instances, the cells of the in vivo assay may be directly evaluated, e.g., for an intrinsic response to a pharmacological agent. In some instances, the host animal of the in vivo assay may be evaluated as an indirect measurement of the response of the cells to the pharmacological agent.

In certain embodiments, the subject disclosure includes screening cells derived according to the methods described herein as a method of therapy of an animal model of disease and/or a human disease. Methods of screening cells derived according to the methods described herein as a method of therapy may be, in some instances, performed according to those methods described below regarding using such cells in therapeutic protocols.

In certain embodiments, the subject disclosure includes screening cells derived according to the methods described herein introduced to a host animal as a method of directly evaluating the cells or particular cellular behaviors, e.g., due to an introduced genetic modification or a naturally derived mutation. In one embodiment, genetically modified cells, e.g., having at least one modified genomic locus, derived according to the methods described herein may be introduced into a host animal and the ability of the cells to differentiate into a particular tissue or cell type may be evaluated. In another embodiment, genetically modified cells derived according to the methods described herein may be introduced into a host animal and the behavior of the cells within the host animal and/or within a tissue of the host animal may be evaluated. In another embodiment, cells derived from a donor organism having a particular mutation or phenotype and lineage restricted according to the methods described herein may be introduced into a host animal and the behavior of the cells within the host animal and/or within a tissue of the host animal may be evaluated, including, e.g., the ability of the cells to differentiate into one or more tissue or cell types. The cells may be introduced into the host animal in a autologous graft, an allograft, or a xenograft such that the introduced cells may be derived from the host animal, a separate donor of the same species as the host animal, or a separate donor of a different species as compared to the host animal, respectively.

### Therapy

Aspects of the disclosure include methods for lessening the symptoms of and/or ameliorating a dysfunction in a mesodermal cell type or a disease of mesodermal origin, herein referred to as mesodermal dysfunction or disorder. Non-limiting examples of mesodermal cell types and/or tissues of mesoderm origin that may be subject to disease or dysfunction that may be treated according to the method described herein include but are not limited to epicardial cells and cardiac pericytes, and the like.

Treatment methods described herein include therapeutic treatments, in which the subject is inflicted prior to administrationand prophylactic treatmentsIn some embodiments, the subject has an increased likelihood of becoming inflicted or is suspected of having an increased likelihood of becoming inflicted (e.g., relative to a standard, e.g., relative to the average individual, e.g., a subject may have a genetic predisposition to mesodermal dysfunction or disorder and/or a family history indicating increased risk of mesodermal dysfunction or disorder), in which case the treatment can be a prophylactic treatment. In some embodiments, the individual to be treated is an individual with mesodermal dysfunction or disorder. As used herein “mesodermal dysfunction or disorder” includes any form of dysfunction of a mesodermal derived tissue or cell type. Any and all forms of mesodermal dysfunction, whether treated or untreated, or resulting from any primary condition, whether treated or untreated, are suitable mesodermal dysfunctions or disorders to be treated by the subject methods described herein.

In some instances, the treatment methods described herein include the alleviation or reduction or prevention of one or more symptoms of mesodermal dysfunction or disorder. Symptoms of mesodermal dysfunction or disorder will vary, may be infrequent, occasional, frequent, or constant.

The methods of treatment described herein include administering a therapeutically effective amount of a population, e.g., an essentially homogenous population, of epicardial cells or cardiac pericytes, to a subject in need thereof.

The effective amount administered varies depending upon the goal of the administration, the health and physical condition of the individual to be treated, age, the taxonomic group of individual to be treated (e.g., human, non-human primate, primate, etc.), the degree of resolution desired (e.g., the amount of alleviation or reduction of symptoms), the formulation of the cell composition, the treating clinician's assessment of the medical situation, and other relevant factors.

A “therapeutically effective dose” or “therapeutic dose” is an amount sufficient to affect desired clinical results (i.e., achieve therapeutic efficacy) or reduce, alleviate, or prevent symptoms to a desired extent as determined by the patient or the clinician. A therapeutically effective dose can be administered in one or more administrations. For purposes of this disclosure, a therapeutically effective dose of cells (e.g., cardiac pericytes) and/or composition is an amount that is sufficient, when administered to (e.g., transplanted into) the individual, to palliate, ameliorate, stabilize, reverse, prevent, slow or delay the progression of the disease state.

In some embodiments, a therapeutically effective dose of cells (e.g. cardiac pericytes, epicardial cells, etc.) is one cell or more (e.g., 1×10² or more, 5×10² or more, 1×10³ or more, 5×10³ or more, 1×10⁴ cells, 5×10⁴ or more, 1×10⁵ or more, 5×10⁵ or more, 1×10⁶ or more, 2×10⁶ or more, 5×10⁶ or more, 1×10⁷ cells, 5×10⁷ or more, 1×10⁸ or more, 5×10⁸ or more, 1×10⁹ or more, 5×10⁹ or more, or 1×10¹⁰ or more).

In some embodiments, a therapeutically effective dose of cells is in a range of from 1×10³ cells to 1×10¹⁰ cells (e.g., from 5×10³ cells to 1×10¹⁰ cells, from 1×10⁴ cells to 1×10¹⁰ cells, from 5×10⁴ cells to 1×10¹⁰ cells, from 1×10⁵ cells to 1×10¹⁰ cells, from 5×10⁵ cells to 1×10¹⁰ cells, from 1×10⁶ cells to 1×10¹⁰ cells, from 5×10⁶ cells to 1×10¹⁰ cells, from 1×10⁷ cells to 1×10¹⁰ cells, from 5×10⁷ cells to 1×10¹⁰ cells, from 1×10⁸ cells to 1×10¹⁰ cells, from 5×10⁸ cells to 1×10¹⁰, from 5×10³ cells to 5×10⁹ cells, from 1×10⁴ cells to 5×10⁹ cells, from 5×10⁴ cells to 5×10⁹ cells, from 1×10⁵ cells to 5×10⁹ cells, from 5×10⁵ cells to 5×10⁹ cells, from 1×10⁶ cells to 5×10⁹ cells, from 5×10⁶ cells to 5×10⁹ cells, from 1×10⁷ cells to 5×10⁹ cells, from 5×10⁷ cells to 5×10⁹ cells, from 1×10⁸ cells to 5×10⁹ cells, from 5×10⁸ cells to 5×10⁹, from 5×10³ cells to 1×10⁹ cells, from 1×10⁴ cells to 1×10⁹ cells, from 5×10⁴ cells to 1×10⁹ cells, from 1×10⁵ cells to 1×10⁹ cells, from 5×10⁵ cells to 1×10⁹ cells, from 1×10⁶ cells to 1×10⁹ cells, from 5×10⁶ cells to 1×10⁹ cells, from 1×10⁷ cells to 1×10⁹ cells, from 5×10⁷ cells to 1×10⁹ cells, from 1×10⁸ cells to 1×10⁹ cells, from 5×10⁸ cells to 1×10⁹, from 5×10³ cells to 5×10⁸ cells, from 1×10⁴ cells to 5×10⁸ cells, from 5×10⁴ cells to 5×10⁸ cells, from 1×10⁵ cells to 5×10⁸ cells, from 5×10⁵ cells to 5×10⁸ cells, from 1×10⁶ cells to 5×10⁸ cells, from 5×10⁶ cells to 5×10⁸ cells, from 1×10⁷ cells to 5×10⁸ cells, from 5×10⁷ cells to 5×10⁸ cells, or from 1×10⁸ cells to 5×10⁸ cells).

In some embodiments, the concentration of cells (e.g., cardiac pericytes, epicardial cells, etc.) to be administered is in a range of from 1×10⁵ cells/ml to 1×10⁹ cells/ml (e.g., from 1×10⁵ cells/ml to 1×10⁸ cells/ml, from 5×10⁵ cells/ml to 1×10⁸ cells/ml, from 5×10⁵ cells/ml to 5×10⁷ cells/ml, from 1×10⁶ cells/ml to 1×10⁸ cells/ml, from 1×10⁶ cells/ml to 5×10⁷ cells/ml, from 1×10⁶ cells/ml to 1×10⁷ cells/ml, from 1×10⁶ cells/ml to 6×10⁶ cells/ml, or from 2×10⁶ cells/ml to 8×10⁶ cells/ml).

In some embodiments, the concentration of cells to be administered is 1×10⁵ cells/ml or more (e.g., 1×10⁵ cells/ml or more, 2×10⁵ cells/ml or more, 3×10⁵ cells/ml or more, 4×10⁵ cells/ml or more, 5×10⁵ cells/ml or more, 6×10⁵ cells/ml or more, 7×10⁵ cells/ml or more, 8×10⁵ cells/ml or more, 9×10⁵ cells/ml or more, 1×10⁶ cells/ml or more, 2×10⁶ cells/ml or more, 3×10⁶ cells/ml or more, 4×10⁶ cells/ml or more, 5×10⁶ cells/ml or more, 6×10⁶ cells/ml or more, 7×10⁶ cells/ml or more, or 8×10⁶ cells/ml or more).

A therapeutically effective dose of cells may be delivered or prepared and any suitable medium, including but not limited to, e.g., those described herein. Suitable medium for the delivery of a therapeutically effective dose of cells will vary and may depend on, e.g., the type of pluripotent cells from which the effective dose of cells is derived or the type of derived cells of the effective dose. In some instances, a suitable medium may be a basal medium. “Cell medium” as used herein are not limited to liquid media may, in some instances, include non-liquid components or combinations of liquid media and non-liquid components. Non-liquid components that may find use a delivery or preparation medium include those described herein and those known in the art. In some instances, non-liquid components include natural or synthetic extra cellular matric components including but not limited to, e.g., basement membrane matrix components and the like.

In some instances, an effective dose of the cells described herein may be co-administered with one or more additional agents (e.g., prepared in a suitable medium). Additional agents useful in such co-administration include agents that improve the overall effectiveness of the effective dose of cells or decrease the dose of cells necessary to achieve an effect essentially equal to administration of an effective dose of the cells without the additional agent. Non-limiting examples of additional agents that may be co-administered include: conventional agents for treating diseases, additional cell types, pro-survival factors, pro-engraftment factors, functional mobilization agents, and the like.

By pro-survival factors is meant a factor or agent that may be added to culture media, delivery excipient, or storage solution that promotes the survival of a desired cell type. Such pro-survival factors may be general pro-survival factors that generally promote the survival of most cell types or may be specific pro-survival factors that only promote the survival of certain specific cell types. In some instances, pro-survival factors of the subject disclosure include but are not limited to, e.g., Rho-associated kinase (ROCK) inhibitor, pinacidil, allopurinol, uricase, cyclosporine (e.g., low does, i.e., sub-immunosuppressive dose, cyclosporine), ZVAD-fmk, pro-survival cytokines (e.g., insulin-like growth factor-1 (IGF-1)), extra cellular matrix (ECM) components, hydrogels, matrigel, collagen, gelatin, agarose, alginate, poly(ethylene glycol), hyaluronic acid, etc.

By pro-engraftment factors is meant a factor or agent that may be added to the administered dose or the delivery excipient or the cell storage solution that, upon delivery of the cells into a subject for treatment, increase the engraftment of the administered cells into the tissue targeted for engraftment and therapy. In some instances, pro-engraftment factors include factors that physically retain the administered cells at the delivery site, e.g., the injection site in the case of direct injection to the affected area, including but not limited to, e.g., gels, polymers, and highly viscous liquids that have physical properties that prevent the administered cells from freely diffusing. Such gels, polymers, and highly viscous liquids include but are not limited to e.g., ECM components, hydrogels, matrigel, collagen, gelatin, agarose, alginate, poly(ethylene glycol), and the like.

The terms “co-administration” and “in combination with” include the administration of two or more therapeutic agents either simultaneously, concurrently or sequentially within no specific time limits. In one embodiment, the agents are present in the cell or in the subject's body at the same time or exert their biological or therapeutic effect at the same time. In one embodiment, the therapeutic agents are in the same composition or unit dosage form. In other embodiments, the therapeutic agents are in separate compositions or unit dosage forms. In certain embodiments, a first agent can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapeutic agent.

The cells may be introduced by injection, catheter, intravenous perfusion, or the like. The cells may be frozen at liquid nitrogen temperatures and stored for long periods of time, being capable of use upon thawing. Once thawed, the cells may be expanded by use of growth factors and/or feeder cells or in feeder-free conditions associated with progenitor cell proliferation and differentiation. In some instances, the cells may be administered fresh such that the cells are expanded and differentiated and administer without being frozen.

The cells and/or compositions of this disclosure can be supplied in the form of a pharmaceutical composition, comprising an isotonic excipient or buffer or media prepared under sufficiently sterile conditions for human administration. For general principles in medicinal formulation, the reader is referred to Cell Therapy: Stem Cell Transplantation, Gene Therapy, and Cellular Immunotherapy, by G. Morstyn & W. Sheridan eds, Cambridge University Press, 1996; and Hematopoietic Stem Cell Therapy, E. D. Ball, J. Lister & P. Law, Churchill Livingstone, 2000. Choice of the cellular excipient and any accompanying elements of the composition will be adapted in accordance with the route and device used for administration. The composition may also comprise or be accompanied with one or more other ingredients that facilitate the engraftment or functional mobilization of the cells. Suitable ingredients include matrix proteins that support or promote adhesion of the cells, or complementary cell types.

Cells of the subject methods may be autologously derived. By autologously derived it is meant that the cells are derived from the subject that is to be treated with the cells. The cells may be derived from a tissue sample obtained from the subject including but not limited to, e.g., a blood sample (e.g., a peripheral blood sample), a skin sample, a bone marrow sample, and the like. In some instances, the sample from which cells are derived may be a biopsy or swab, e.g., a biopsy or swab collected to diagnose, monitor, or otherwise evaluate the subject, e.g., diagnose the subject for a mesodermal dysfunction or deficiency, e.g., bone disease or a muscle disease or a cartilage disease or a related condition, or for cell collection. In some instances, the autologous sample from which the cells are derived may be a previously collected and stored sample, e.g., a banked tissue sample, from the subject to be treated, including but not limited to e.g., banked cardiac tissue or cells, banked musculoskeletal tissue or cells, banked reproductive tissue or cells, banked skin tissue or cells, banked bone tissue or cells, banked bone marrow tissue or cells, banked vascular tissue or cells, banked umbilical cord blood tissue or cells, and the like.

In some instances, cells of the subject methods may be non-autologously derived. By non-autologously derived it is meant that the cells are not derived from the subject that is to be treated with the cells. In some instances, non-autologously derived cells may be xeno-derived (i.e., derived from a non-human animal) or allo-derived (i.e., derived from a human donor other than the subject to be treated). Non-autologously derived cells or tissue may be derived from any convenient source of cells or tissue collected by any convenient means.

Whether to use autologously derived or non-autologously derived cells may be determined according to the discretion of the subject's clinician and may depend on, e.g., the health, age, genetic predisposition or other physical state of the subject. In some instances, autologous cells may be preferred, including, e.g., to decrease the risk or immune rejection of the transplanted cells. In some instances, non-autologous cells may be preferred, including, e.g., when the subject has a genetic defect that affects mesodermally derived tissues.

Methods of derivation of pluripotent progenitor cells from an autologous or non-autologous tissue useful in the methods described herein include but are not limited to, e.g., methods of embryonic stem cell derivation and methods of induced pluripotent stem cell derivation. In some instances, methods as described herein may be performed using non-autologous pluripotent progenitor cells previously derived including, e.g., those publically available (e.g., Stanford Cardiovascular Institute Biobank, Stanford, CA) or commercially available (e.g., from Biotime, Inc., Alameda, CA). In some instances, methods as described herein may be performed using newly derived non-autologous pluripotent progenitor cells or newly derived autologous pluripotent progenitor cells including but not limited to, e.g., newly derived embryonic stem cells (ESC) (including, e.g., those derived under xeno-free conditions as described in, e.g., Lei et al. (2007) Cell Research, 17:682-688) and newly derived induced pluripotent stem cells (iPSC). General methods of inducing pluripotency to derive pluripotent progenitor cells are described in, e.g., Rodolfa K T, (2008) Inducing pluripotency, StemBook, ed. The Stem Cell Research Community, doi/10.3824/stembook. 1.22.1 and Selvaraj et al. (2010) Trends Biotechnol, 28 (4) 214-23, the disclosures of which are incorporated herein by reference. In some instances, pluripotent progenitor cells, e.g., iPS cells, useful in the methods described herein are derived by reprogramming and are genetically unmodified, including e.g., those derived by integration-free reprogramming methods, including but not limited to those described in Goh et al. (2013) PLoS ONE 8 (11): e81622; Awe et al (2013) Stem Cell Research & Therapy, 4:87; Varga (2014) Exp Cell Res, 322 (2) 335-44; Jia et al. (2010) Nat Methods, 7 (3): 197-9; Fusaki et al. (2009) Proc Jpn Acad Ser B Phys Biol Sci. 85 (8): 348-62; Shao & Wu, (2010) Expert Opin Biol Ther. 10 (2): 231-42; the disclosures of which are incorporated herein by reference.

In some instances, the derived or obtained pluripotent progenitor cells are prepared, dissociated, maintained and/or expanded in culture prior to being differentiated and/or lineage restricted as described herein.

In some instances, before differentiation or lineage restriction of the pluripotent progenitor cells are dissociated, e.g., to generate a single-cell suspension. In some instances, the dissociation of the pluripotent progenitors is chemical, molecular (e.g., enzyme mediated), or mechanical dissociation. Methods of chemical, molecular, and/or enzyme mediated dissociation will vary and, in some instances, may include but are not limited to the use of, e.g., trypsin, TrypLE Express™, TrypLE Select™, Accutase®, StemPro® (Life Technologies, Inc., Grand Island, NY), calcium- and magnesium-free media, low calcium and magnesium medium, and the like. In some instances, the dissociation media may further include pro-survival factors including but not limited to, e.g., Rho-associated kinase (ROCK) inhibitor, pinacidil, allopurinol, uricase, cyclosporine (e.g., low does, i.e., sub-immunosuppressive dose, cyclosporine), ZVAD-fmk, pro-survival cytokines (e.g., insulin-like growth factor-1 (IGF-1)), Thiazovivin, etc.

In some instances, methods of culturing pluripotent stem cells include xeno-free culture conditions wherein, e.g., human cells are not cultured with any reagents derived from non-human animals. In some instances, methods culturing of pluripotent stem cells include feeder-free culture conditions, wherein the pluripotent stem cells are cultured under conditions that do not require feeder cells and/or in feeder cell free medium, including e.g., commercially available feeder-free mediums, such as, e.g., those available from STEMCELL Technologies, Inc. (Vancouver, BC). In some instances, methods of culturing pluripotent stem cells include culture conditions that include supplemental serum, including supplement of autologously derived serum, as described in Stute et al. (2004) Exp Hematol, 32 (12): 1212-25. In some instances, methods of culturing of pluripotent cells or derivatives thereof include culture conditions that are serum-free, meaning the culture media does not contain animal, mammal, or human derived serum. Serum-free culture conditions may be performed for only a portion of the life of the culture or may performed for the entire life of the culture. In some instances, serum-free culture conditions are used for a particular method step or procedure, e.g., during differentiation, during lineage restriction, prior to or during harvesting, etc. As is known in the art, in some instances, cells may be cultured in two-dimensional or three-dimensional formats (e.g., on non-coated or coated surfaces or within a solid or semi-solid matrix). Instances where two dimensional or three-dimensional culture is appropriate for use in the methods as described herein, e.g., to promote survival or differentiation of a desired cell type, will be readily apparent to the ordinary skilled artisan. In some instance, the pluripotent progenitor cell media include one or more pro-survival factors, e.g., including those described herein. General methods of culturing human pluripotent progenitor cells are described in, e.g., Freshney et al. (2007) Culture of human stem cells, Wiley-Interscience, Hoboken, NJ and Borowski et al. (2012) Basic pluripotent stem cell culture protocols, StemBook, ed. The Stem Cell Research Community, StemBook, doi/10.3824/stembook, the disclosures of which are incorporated herein by reference.

In some instances, the pluripotent progenitor cells used according to the methods described herein may be genetically unmodified. By “genetically unmodified” is meant that essentially no modification of the genome of the cells transplanted into the subject has been performed. Encompassed within the term genetically unmodified are instances wherein transient genetic modification is performed at some point during the derivation of the cells but essentially no genetic modification persists in the cells that are eventually transplanted into the subject (i.e., the cells are essentially indistinguishable before the transient genetic modification and after the course of the transient modification). Also encompassed within the term genetically unmodified are instances wherein the genome of the cells is not transiently or stably modified, e.g., where the cells are manipulated, e.g., pluripotent progenitors are derived or cells are transformed, without genetic modification (e.g., modification of the nucleotide sequence of the genome) of the cells.

For further elaboration of general techniques useful in the practice of this disclosure, the practitioner can refer to standard textbooks and reviews in cell biology, tissue culture, and embryology. With respect to tissue culture and stem cells, the reader may wish to refer to Teratocarcinomas and embryonic stem cells: A practical approach (E. J. Robertson, ed., IRL Press Ltd. 1987); Guide to Techniques in Mouse Development (P. M. Wasserman et al. eds., Academic Press 1993); Embryonic Stem Cell Differentiation in Vitro (M. V. Wiles, Meth. Enzymol. 225:900, 1993); Properties and uses of Embryonic Stem Cells: Prospects for Application to Human Biology and Gene Therapy (P. D. Rathjen et al., Reprod. Fertil. Dev. 10:31, 1998).

### Systems

Also provided are systems for use in practicing the subject methods. Systems of the subject disclosure may include a cell production system, e.g., for the production of a homogenous or highly pure population of derived mesodermal cell types from pluripotent progenitor cells.

In some instances, the cell production system includes a cell culture chamber or cell culture vessel for the culture of desired cell types. Such cell culture chambers may be configured for the expansion of pluripotent progenitor cells and for the differentiation and/or lineage restriction of such pluripotent progenitor cells into desired cell types, e.g., derived mesodermal cell types and/or differentiated mesodermal cell types. In some instances, the cell culture chamber is also configured for the expansion of mesodermal cell types and/or differentiated mesodermal cell types. In certain embodiments, the cell culture chamber or cell culture vessel may be an open culture system, including but not limited to e.g., tissue culture dishes, tissue culture plates, tissue culture multi-well plates, tissue culture flasks, etc. In certain embodiments, the cell culture chamber or cell culture vessel may be a closed culture system, including e.g., a bioreactor, a stacked tissue culture vessel (e.g., CellSTACK Culture Chambers, Corning, NY). In some instances, culture media and or other factors or agents may be exchanged in and out of the cell culture chamber through the use of one or more pumps (e.g., syringe pumps, peristaltic pumps, etc.) or gravity flow devices. In instances where the cells are cultured under sterile conditions, the culture system may allow for the sterile exchange of culture media, e.g., through the use of sterile tubing connected, sealed, and reconnected through the use of a sterile devices, including but not limited to, e.g., a sterile tube welder and/or a sterile tube sealer. The cell culture system may be configured to control certain environmental conditions, including but not limited to e.g., temperature, humidity, light exposure, air composition (e.g., oxygen levels, carbon dioxide levels, etc.) to achieve the conditions necessary for expansion and/or differentiation of desired cell types. In some instances, the cell culture chamber may include a cell culture vessel that includes one or more patterned cell culture substrates or one or more arrays of patterned cell culture substrates as described herein.

The cell culture chamber may be configured for the production of cells for clinical use, e.g., according to current good manufacturing practice (cGMP) compliant cell culture practices, including the methods and configurations described in e.g., Fekete et al. PLoS ONE (2012) 7 (8): e43255; Pham et al. (2014) J Trans Med 12:56; Gastens et al. (2007) Cell Transplant 16 (7): 685-96; Fernandes et al. (2013) Stem Cell Bioprocessing: For Cellular Therapy, Diagnostics and Drug Development, Burlington, Oxford: Elsevier Science: Woodhead Publishing, the disclosures of which are incorporated herein by reference.

The cell production system may, in some instances, by computer-controlled and/or automated. Automated and/or computer-controlled cell production systems may include a “memory” that is capable of storing information such that it is accessible and retrievable at a later time or date by a computer. Any convenient data storage structure may be chosen, based on the means used to access the stored information. In certain aspects, the information may be stored in a “permanent memory” (i.e., memory that is not erased by termination of the electrical supply to a computer or processor) or “non-permanent memory”. Computer hard-drive, CD-ROM, floppy disk, portable flash drive and DVD are all examples of permanent memory. Random Access Memory (RAM) is an example of non-permanent memory. A file in permanent memory may be editable and re-writable.

In certain instances, a computer-controlled and/or automated cell culture system may include a module or program stored in memory for production of cells according to the methods described herein. Such a module may include instructions for the administration of induction agent and/or induction compositions, e.g., at particular timing intervals or according to a particular schedule, in order to generate a desired mesodermally derived cell type. In some instances, such a computer module may further include additional modules for routine cell culture tasks including but not limited to, e.g., monitoring and record keeping, media changes, environmental monitoring, etc.

Systems of the present disclosure include components and/or devices for delivering cells produced according to the methods described herein to a subject in need thereof. For example, in some instances a system for treating a subject with a mesodermal derived tissue dysfunction or deficiency includes a cell injection system for delivering cells in a carrier, with or without optional adjuvants, to a desired injection site, including diseased tissue, adjacent to diseased tissue, and/or within, on or near a dysfunctioning organ. Such systems utilize known injection devices (e.g., including but not limited to needles, bent needles, cannulas, syringes, pumps, infusion devices, diffusion devices, etc.) and techniques (e.g., including but not limited to intramuscular injection, subcutaneous injection, device-guided injection, etc.). In some instances, a device or technique used for the delivery of a cell scaffold or other bioengineered device may be configured or adapted for use in a cell delivery system for use in delivering cells derived according to the methods described herein.

In addition to the above-described components systems of the subject disclosure may include a number of additional components, such as data output devices, e.g., monitors and/or speakers, data input devices, e.g., interface ports, keyboards, etc., fluid handling components, power sources, controllers, etc.

### Compositions and Kits

Also provided are compositions and kits for use in the subject methods. The subject compositions and kits include any combination of components for performing the subject methods. In some embodiments, a composition can include, but is not limited to and does not require, the following: cell dissociation agents and/or media, cell reprogramming agents and/or media, pluripotent progenitor cells, cell culture agents and/or media, cell differentiation agents and/or media; lineage restriction agents (e.g., induction agents) and/or media; conventional agents for treating diseases and/or dysfunctions of mesodermally derived tissues, non-mesodermally derived cell types, pro-survival factors, pro-engraftment factors, functional mobilization agents and any combination thereof.

In some embodiments, a kit can include, but is not limited to and does not require, the following: any of the above described composition components, a sample collection container, a sample collection device (e.g., a sample collection container that includes a sample enrichment mechanism including, e.g., a filter), a tissue collection device (e.g., a biopsy device), a tissue dissociation device, a cell culture vessel, a cell production system; and any combination thereof.

In some embodiments, a kit can include, but is not limited to and does not require, a cell delivery system and/or a cell injection system configured for delivery of cells derived according to the methods described herein. For example, a kit may include a cell injection system configured for injection or delivery of cells into a desired area of the subject in order to effectively treat the subject for a mesodermally derived tissue dysfunction or deficiency, e.g., through delivery of cells to the mesodermally derived tissue. Such kits may include a cell delivery or injection system, as described herein, including individual components of such systems in assembled or unassembled form. In some instances, cells derived according to the methods described herein may be “preloaded” into a cell injection or delivery system such that the system is provided in a “ready-to-use” configuration. In other instances, a cell injection or delivery system may be provided in an “unloaded” configuration such that cells derived according to the methods described herein must be loaded into the system, with any desired carrier or vehicle, prior to use.

In addition to the above components, the subject kits may further include (in certain embodiments) instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, and the like. Yet another form of these instructions is a computer readable medium, e.g., diskette, compact disk (CD), flash drive, and the like, on which the information has been recorded. Yet another form of these instructions that may be present is electronic, e.g., a website address which may be used via the internet to access the information at a removed site.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g., amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., room temperature (RT); base pairs (bp); kilobases (kb); picoliters (pl); seconds (s or see); minutes (m or min); hours (h or hr); days (d); weeks (wk or wks); nanoliters (nl); microliters (ul); milliliters (ml); liters (L); nanograms (ng); micrograms (ug); milligrams (mg); grams ((g), in the context of mass); kilograms (kg); equivalents of the force of gravity ((g), in the context of centrifugation); nanomolar (nM); micromolar (μM), millimolar (mM); molar (M); amino acids (aa); kilobases (kb); base pairs (bp); nucleotides (nt); intramuscular (i.m.); intraperitoneal (i.p.); subcutaneous (s.c.); and the like. Methods are provided for producing mesodermal progenitor cell types, epicardial cells, and terminally differentiated cardiac pericytes. Also provided are methods of screening for cellular responses and treating a subject for a condition using the produced epicardial cells and/or terminally differentiated cardiac pericytes. The instant disclosure also provides systems and kits for producing mesodermal cell types and/or screening for cellular responses and/or treating subjects with such mesodermal cell type.

### Stepwise Generation of Human Induced Pluripotent Stem Cell-Derived Cardiac Pericytes to Model Coronary Microvascular Dysfunction

Here, we report a stepwise approach to generate first-of-its-kind CPs from human induced pluripotent stem cells (IPSCs), which were shown to transcriptionally and functionally resemble their primary counterparts.

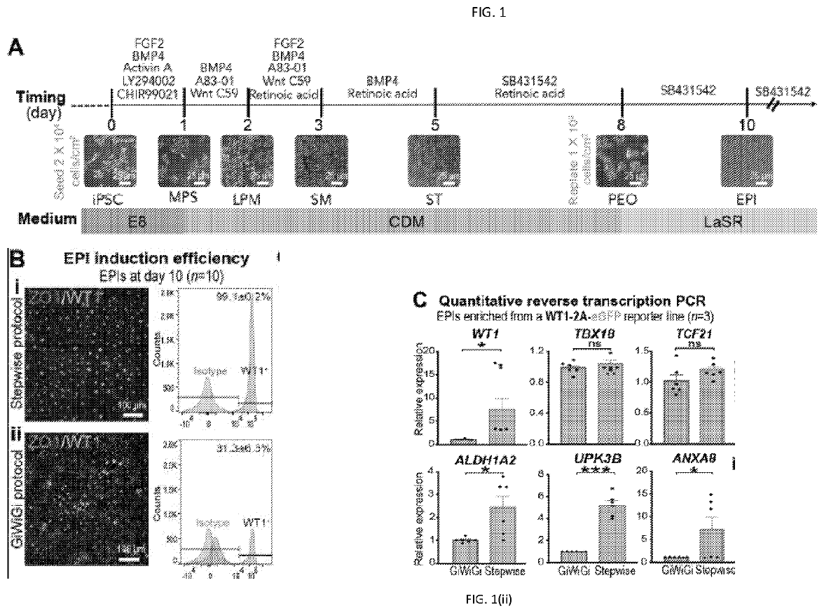

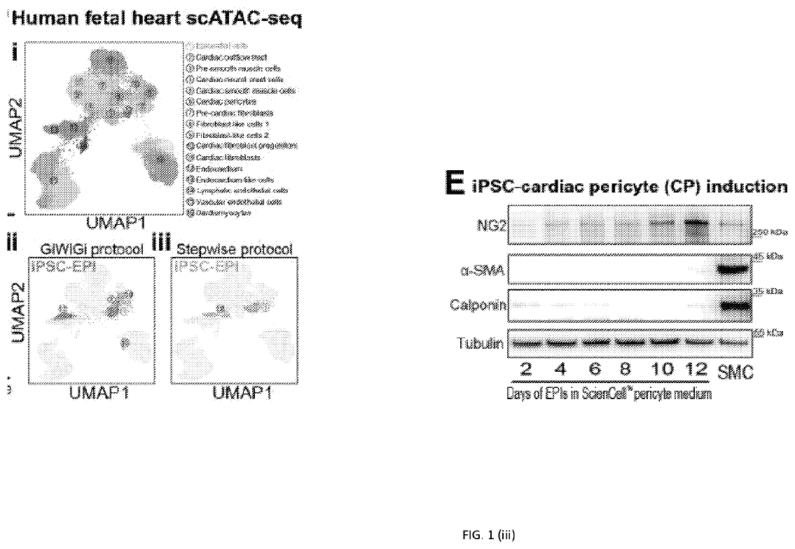

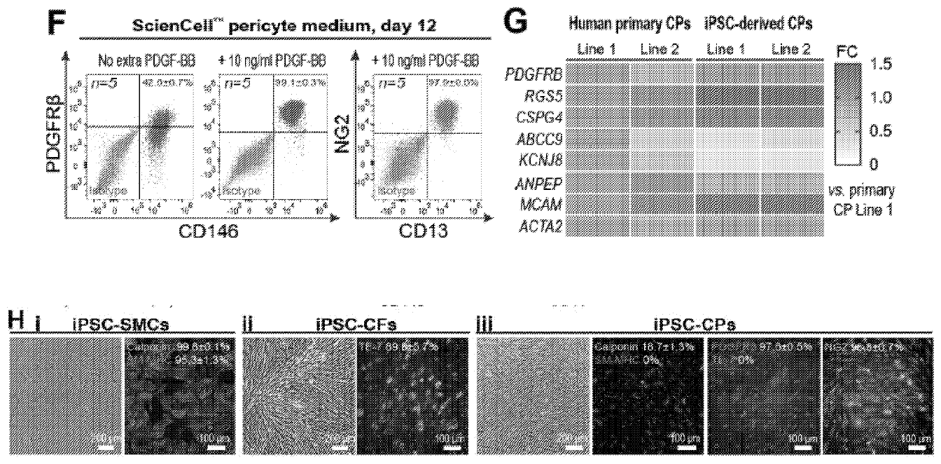

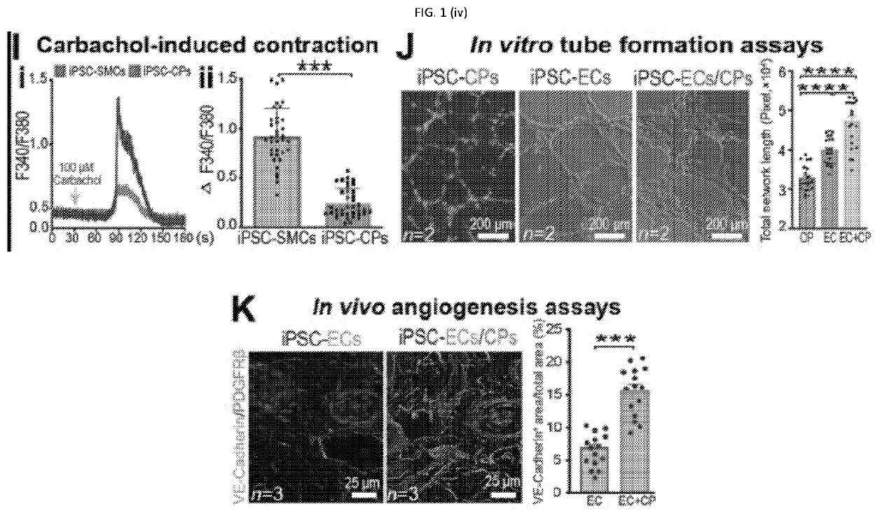

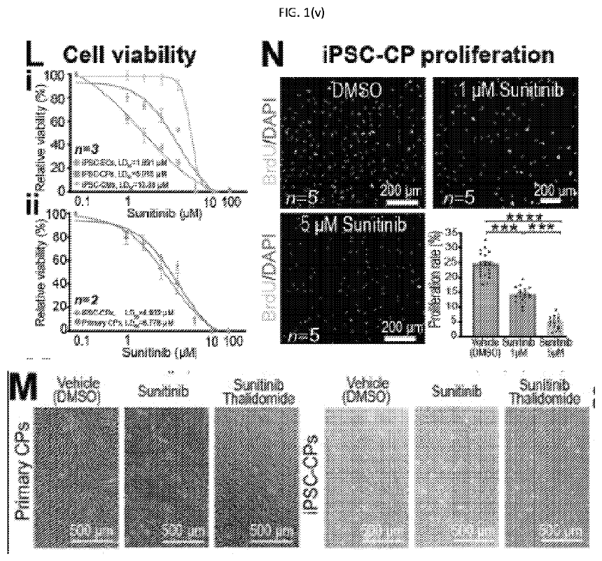

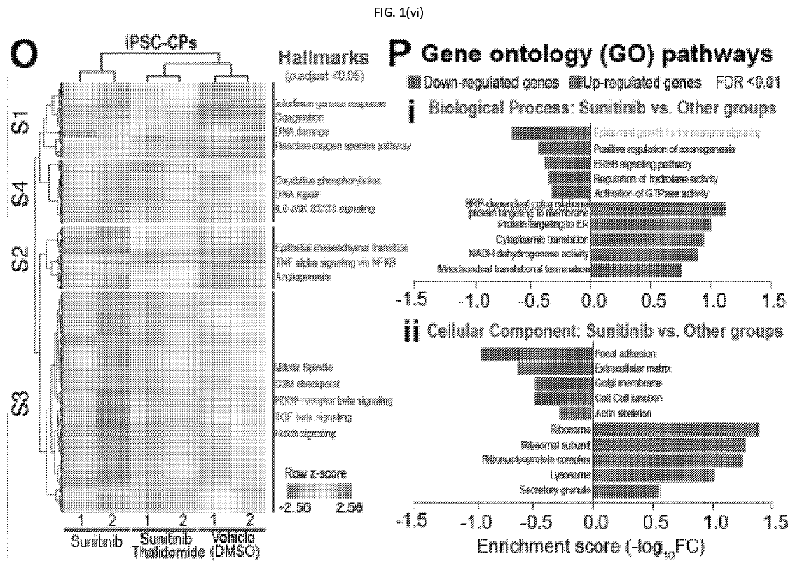

To derive pure iPSC-CPs, we first generated epicardial cells (EPIs), the predominant progenitor cells giving rise to CPs, in a stepwise fashion (FIG. 1A). We found that temporal activation or inhibition of key morphogens at different differentiation stages enabled the generation of pure mesodermal progenitors and EPIs (FIG. 1B). In contrast, using an extant protocol that only manipulated Wnt signaling throughout the entire differentiation process (referred to as GiWiGi protocol), we observed a large variation of EPI induction efficiency (ranging from 3%-87%) even among iterative differentiations (FIG. 1B). Moreover, our stepwise protocol generated more mature EPIs (ALDH1A2, UPK3B, and ANXA8) than did the GiWiGi protocol (FIG. 1C). Finally, we performed single-cell ATAC sequencing (scATAC-seq) on stepwise and GiWiGi EPIs and projected them onto human fetal heart scATAC-seq data based on their chromatin landscape similarities. Our data showed that >30% of GiWiGi EPIs acquired a fibroblast fate (FIG. 1D), precluding the generation of pure CPs for cell type-specific studies.

In contrast, stepwise EPIs overlapped with primary EPIs and endocardial cells, two progenitors that can give rise to CPs (FIG. 1D).

Next, we differentiated stepwise EPIs in a commercial pericyte medium and observed that it took at least 12 days to generate iPSC-CPs (FIG. 1E) with low efficiency (FIG. 1F). Since pericyte-endothelial cell (EC) crosstalk and platelet-derived growth factor receptor (PDGFR) signaling are critical for pericyte development, we successfully generated pure iPSC-CPs (PDGFRβ+/CD146+/NG2+/CD13+) with exogenous PDGF-BB, a ligand predominantly secreted by ECs (FIG. 1F). Next, we confirmed comparable expression levels of cell markers between primary and iPSC-CPs using quantitative reverse transcription PCR (FIG. 1G). Finally, we showed that iPSC-CPs are negative for smooth muscle cell (SMC) markers and not reactive to an anti-fibroblast antibody TE-7 (FIG. 1H), further confirming their cell type specificity.

Next, we examined the relative cell contraction capacities of iPSC-CPs versus iPSC-SMCs by measuring intracellular calcium transients after carbachol treatment. While both cell types showed a stereotypical contraction pattern to carbachol, iPSC-CPs had a much smaller calcium amplitude, indicating weaker contraction due to lower intracellular calcium increase and lower expression levels of contractile proteins (FIG. 11). We also tested the pro-angiogenic potentials of iPSC-CPs. We observed that iPSC-CPs not only spontaneously formed a tubular network in vitro, but also improved tube formation in vitro and neovessel maturation in vivo when they were mixed with iPSC-ECs (FIGS. 1J and 1K).

A previous study reported that CPs were the primary targets of sunitinib-induced coronary microvascular abnormalities and cardiac dysfunction in mice. To validate whether our iPSC-CPs can recapitulate this phenotype, we treated iPSC-CPs, iPSC-ECs, and iPSC-cardiomyocytes (CMs) with a wide dose range of sunitinib. In line with the in vivo findings, we observed that iPSC-CPs were more sensitive than iPSC-CMs to sunitinib-induced cell death (FIG. 1Li). In contrast, primary and iPSC-CPs showed a similar cytotoxicity profile to sunitinib treatment (FIG. 1Lii and 1M). Sunitinib significantly suppressed iPSC-CP proliferation in a dose-dependent manner (FIG. 1N). Notably, thalidomide, a drug that can rescue mice from sunitinib-induced CP loss and cardiac dysfunction, profoundly rescued CP death (FIG. 1M) and restored genes associated with cell cycle, pericyte function, and DNA damage to a baseline level (FIGS. 10 and 1P]). Because the precise mechanisms by which sunitinib-induced CP cytotoxicity remain elusive, our iPSC model, if combined with high throughput screening tools, can help identify novel therapeutics to prevent cardiotoxicity induced by sunitinib or other agents that primarily inhibit PDGFR signaling.

In summary, our human iPSC-CPs represent a novel model system essential to understanding coronary microvascular dysfunction. The high resemblance between iPSC-CPs and their in vivo counterparts will help researchers understand genetic or environmental factor-induced coronary microvasculature malformation, spur the development of more effective pro-angiogenic cell therapies for patients experiencing myocardial infarction, and spark novel approaches for drug toxicity evaluation and discovery.

Human heart tissue samples, animals, and iPSCs used in the study were approved by the Stanford Institutional Review Board (#42056), Administrative Panel on Laboratory Animal Care (#34262), and Stem Cell Research Oversight Committee (#563), respectively. All scATAC-seq data can be accessed at GSE181346 and RNA-seq data at GSE210652. Raw data can be made available upon request from the corresponding author.

### REFERENCES

Chintalgattu V, Rees ML, Culver JC, Goel A, Jiffar T, Zhang J, Dunner K, Jr., Pati S, Bankson J A, Pasqualini R, et al. Coronary microvascular pericytes are the cellular target of sunitinib malate-induced cardiotoxicity. Sci Transl Med. 2013; 5: 187ra169. doi: 10.1126/scitranslmed.3005066
Shen M, Liu C, Wu JC. Generation of embryonic origin-specific vascular smooth muscle cells from human induced pluripotent stem cells. Methods Mol Biol (Clifton, NJ). 2022; 2429:233-246. doi: 10.1007/978-1-0716-1979-7_15
Bao X, Lian X, Hacker TA, Schmuck E G, Qian T, Bhute V J, Han T, Shi M, Drowley L, Plowright A, et al. Long-term self-renewing human epicardial cells generated from pluripotent stem cells under defined xeno-free conditions. Nat Biomed Eng. 2016; 1. doi: 10.1038/s41551-016-0003
Ameen M, Sundaram L, Banerjee A, Shen M, Kundu S, Nair S, Scherbina A, Gu M, Wilson K, Varadarajan A, et al. Integrative single-cell analysis of cardiogenesis identifies developmental trajectories and non-coding mutations in congenital heart disease. bioRxiv. 2022:73. doi: 10.1101/2022.06.29.498132
Chen Q, Zhang H, Liu Y, Adams S, Eilken H, Stehling M, Corada M, Dejana E, Zhou B, Adams R H. Endothelial cells are progenitors of cardiac pericytes and vascular smooth muscle cells. Nat Commu. 2016; 7:12422. doi: 10.1038/ncomms12422

### Materials and Methods

### Compound Stock Concentrations

TGF activator: Activin A (10 μg/ml)
TGFβ inhibitor: A-83-01 (1 mM) & SB431542 (10 mM)
WNT activator: CHIR99021 (10 mM)
WNT inhibitor: Wnt-C59 (1 mM)
PI3K inhibitor: LY294002 (10 mM)
BMP4: 100 μg/ml FGF2: 100 g/ml
Retinoic acid: 10 mM Ascorbic acid: 25 mg/ml

### Basal Medium Compositions

E8 medium. Essential 8™ Medium is a xeno-free and feeder-free medium specially formulated for the growth and expansion of human pluripotent stem cells (PSCs). Originally developed by Guokai Chen et al. in the laboratory of James Thomson (published as ‘E8’) and validated by Cellular Dynamics International, Essential 8TM Medium has been extensively tested and proved to maintain pluripotency in multiple iPSC lines.

Basal chemically defined medium (CDM), 500 ml: consisting of 240 ml of IMDM (50% vol/vol), 240 ml of Ham's F-12 Nutrient Mix (50% vol/vol), 5 ml of chemically defined lipid concentrate (1% vol/vol); 5 ml of Glutamax (2 mM), 5 ml of PVA (1 mg/ml), 250 μl of transferrin (15 μg/ml), and 20 μl of monothioglycerol (450 μM). Basal CDM is sterile filtered after preparation and can be stored for up to 4 weeks at 4° C.

LaSR medium, 500 ml: consisting of 500 ml of advanced DMEM/F-12, 2 ml of ascorbic acid (100 μg/ml) and 6.5 ml of GlutaMax (100×).

As shown in FIG. 1A, a step-wise method is provided for generating a population of epicardial cells, which are then differentiated to cardiac pericytes. The days of culture and medium are shown in the figure.

Day 1. iPSC to Mid-primitive streak, MPS. Aspects of the disclosure relate to producing mid-primitive streak cells through contacting a population of pluripotent progenitor cells with a mid-primitive streak induction composition. As described herein, mid primitive streak induction compositions may vary and generally comprises effective amounts of a TGF-beta pathway activator; a Wnt pathway activator; an FGF pathway activator; a BMP pathway activator; and a PI3K pathway inhibitor. An exemplary MPS induction medium comprises 10 ng/ml Activin A; 6 μM CHIR; 20 ng/ml FGF2; 50 ng/ml BMP4; 2 μM LY294002 in CDM.

Day 2. MPS to Lateral plate mesoderm, LPM. Aspects of the disclosure relate to producing lateral plate mesodermal cells through contacting a population of mid-primitive streak cells with a lateral plate mesoderm induction composition. As described herein, lateral plate mesoderm induction compositions may vary and generally comprise effective amounts of a TGF-beta pathway inhibitor; a Wnt pathway inhibitor; and a BMP pathway activator. An exemplary LPM induction medium comprises 1 μM A8301; 30 ng/ml BMP4; 1 μM C59 in CDM.

Day 3. LPM to Splanchnic mesoderm, SM. Aspects of the disclosure relate to producing splanchnic mesodermal cells through contacting a population of lateral plate meosodermal cells with a splanchnic mesoderm induction composition. As described herein, splanchnic mesoderm induction compositions may vary and generally include effective amounts of a TGF-beta pathway inhibitor; a Wnt pathway inhibitor; a BMP pathway activator; an FGF pathway activator; and a retinoic acid pathway activator. An exemplary SM induction medium comprises 1 μM A8301+30 ng/ml BMP4+1 μM C59+20 ng/ml FGF2+2 μM RA in CDM.

Days 4-5. SM to Septum Transversum, ST. Aspects of the disclosure relate to producing septum transversum cells through contacting a population of splanchnic mesodermal cells with a septum transversum induction composition. As described herein, septum transversum induction compositions may vary and generally include effective amounts of a BMP pathway activator; and a retinoic acid pathway activator. An exemplary ST induction medium comprises 40 ng/ml BMP4+2 μM RA in CDM.

Days 5-8. ST to Proepicardium organ, PEO. Aspects of the disclosure relate to producing proepicardial cells through contacting a population of septum transversum cells with a proepicardium organ induction composition. As described herein, proepicardium organ induction compositions may vary and generally include effective amounts of a retinoic acid pathway activator; and a TGF-beta pathway inhibitor in LaSR medium or equivalent. An exemplary PEO induction medium comprises 2 μM RA+1 μM A83-01 in LaSR medium.

Days 8-10. PEO to Epicardium, EPI. Aspects of the disclosure relate to producing epicardial cells through contacting a population of proepicardial cells with an epicardium induction composition. As described herein, epicardium induction compositions may vary and may generally include effective amounts of a TGF-beta pathway inhibitor in LaSR medium or equivalent. An exemplary EPI induction medium comprises 1 μM A83-01 in LaSR medium

Day 11. EPI to Cardiac pericyte, CP. Aspects of the disclosure relate to producing cardiac pericytes through contacting a population of epicardial cells with a cardiac pericyte composition. As described herein, cardiac pericyte induction compositions may vary and generally comprise effective amounts of a PDGF pathway activator in a commercial pericyte medium (ScienCell cat #1201). An exemplary CP induction medium comprises 10 ng/ml PDGF-BB in pericyte medium.

The preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of the present invention is embodied by the appended claims.

1. A stepwise method of producing a population of epicardium cells, the method comprising:
(a) contacting a population of pluripotent stem cells in culture with mid-primitive streak (MPS) induction media to induce a population of MPS cells;
(b) contacting the population of MPS cells with lateral plate mesoderm (LPM) induction medium to induce a population of LPM cells;
(c) contacting the population of LPM cells with splanchnic mesoderm (SM) induction medium to induce a population of SM cells;
(d) contacting the population of SM cells with septum transversum (ST) induction medium to induce a population of ST cells;
(e) contacting the population of ST cells with proepicardium organ (PEO) induction medium to induce a population of PEO cells;
(f) contacting the population of PEO cells with epicardium (EPI) induction medium to induce a population of EPI cells.

2. The method of claim 1, wherein the population of epicardium cells are substantially pure.

3. The method of claim 1, further comprising:
(g) contacting the population of EPI cells with pericyte induction medium to induce a population of cardiac pericytes.

4. The method of claim 1, wherein steps (a) and (d) are for a period of about 24 hours each.

5. The method of claim 1, wherein steps (a) and (d) are for a period consisting essentially of 24 hours.

6. The method of claim 1, wherein step (b) is for a period of about 48 hours.

7. The method of claim 1, wherein step (b) is for a period consisting essentially of 48 hours.

8. The method of claim 1, wherein step (c) is for a period of about 72 hours.

9. The method of claim 1 wherein step (c) is for a period consisting essentially of 72 hours.

10. The method of claim 1, wherein steps (e) and (f) are for a period of about 1 to 3 days.

11. The method of claim 1, wherein step (g) is for a period of about 6 to about 12 days.

12. The method of claim 1, wherein the mid-primitive streak induction medium comprises an effective dose of a TGF-beta pathway activator; a Wnt pathway activator; an FGF pathway activator; a BMP pathway activator; and a PI3K pathway inhibitor.

13. The method of claim 1, wherein the lateral plate mesoderm induction medium comprises an effective dose of a TGF-beta pathway inhibitor; a Wnt pathway inhibitor; and a BMP pathway activator.

14. The method of claim 1, wherein the splanchic mesoderm induction medium comprises an effective dose of a TGF-beta pathway inhibitor; a Wnt pathway inhibitor; a BMP pathway activator; an FGF pathway activator; and a retinoic acid pathway activator.

15. The method of claim 1, wherein the septum transversum induction medium comprises an effective dose of a BMP pathway activator; and a retinoic acid pathway activator.

16. The method of claim 1, wherein the proepicardium organ induction medium comprises an effective dose of a retinoic acid pathway activator; and a TGF-beta pathway inhibitor in LaSR medium or equivalent.

17. The method of claim 1, wherein the epicardium induction medium comprises an effective dose of a TGF-beta pathway inhibitor in LaSR medium or equivalent.

18. The method of claim 1, wherein the pericyte induction medium comprises an effective dose of a PDGF pathway activator in pericyte medium.

19. A substantially pure population of epicardium or pericyte cells obtained by the methods of claim 1.

20. A method of screening for a cellular response, the method comprising:
a) contacting a population of cells of claim 19 with a pharmacological agent; and
b) evaluating the population of cells for a cellular response induced by the pharmacological agent.

21. (canceled)
